(19) United States
(12) Patent Application Publication
(10) Pub. No.: US 2016/0209415 A1
(43) Pub. Date: Jul. 21, 2016

(54) METHOD TO PREDICT OR DIAGNOSE A COLORECTAL CANCER

(71) Applicant: Poochon Scientific LLC, Frederick, MD (US)

(72) Inventors: Jian-Jiang Hao, Adamstown, MD (US); Jianwei Zhu, Nantong (CN)

(21) Appl. No.: 14/987,027

(22) Filed: Jan. 4, 2016

Related U.S. Application Data
(60) Provisional application No. 62/105,642, filed on Jan. 20, 2015.

(51) Int. Cl. G01N 33/574

(52) U.S. Cl. CPC G01N 33/57419; G01N 2570/00; G01N 2333/705; G01N 2333/948; G01N 2333/4703; G01N 2333/71; G01N 2333/912; G01N 2333/8121; G01N 2333/4725

(57) ABSTRACT

It discloses eleven biomarkers for colorectal cancer, wherein the disclosure provides a method and a kit useful for determining if a subject has an increased risk having a colorectal disease or disorder.

This application claims priority to U.S. patent application Ser. No. 62/105,642 filed 20 Jan. 2015.

### TECHNICAL FIELD OF THE INVENTION

The present invention relates generally to methods and diagnostic kits for predicting or diagnosing colorectal cancer by measuring biomarkers.

### BACKGOUND OF THE INVENTION

Colorectal cancer (also known as colon cancer, rectal cancer or bowel cancer) is the development of cancer in the colon or rectum (parts of the large intestine). It is due to the abnormal growth of cells that have the ability to invade or spread to other parts of the body. Signs and symptoms may include blood in the stool, a change in bowel movements, weight loss, and feeling tired all the time.

Most colorectal cancers are due to lifestyle factors and increasing age, with only a small number of cases due to inherited genetic disorders. Risk factors include diet, obesity, smoking, and not enough physical activity. Dietary factors that increase the risk include red and processed meat, as well as alcohol. Another risk factor is inflammatory bowel disease, which includes Crohn's disease and ulcerative colitis. Some of the inherited conditions that can cause colorectal cancer include: familial adenomatous polyposis and hereditary non-polyposis colon cancer; however, these represent less than 5% of cases. It typically starts as a benign tumor which over time becomes cancerous.

Diagnosis of colorectal cancer is via sampling of areas of the colon suspicious for possible tumor development typically done during colonoscopy or sigmoidoscopy, depending on the location of the lesion. However, no biomarkers which would provide information for developmental stages of colorectal cancer and guide the treatment have identified. The present invention discloses at least 11 biomarkers for colorectal cancer, and develops diagnostic process and kit for diagnosis with the biomarkers.

### SUMMARY OF THE INVENTION

Extensive genomic characterizations of human cancers have provided the most compelling demonstrations of function-altering mutations and of ongoing genomic instability during tumor progression. However, it is not fully understood how dozens of mutated tumor suppressor genes and oncogenes drive cancers. As proteins link genotypes to phenotypes, alterations in the proteome of cancer cells shall play crucial roles during carcinogenesis. The present invention shows the first comprehensive map of the colorectal cancer proteome and its abnormal features by proteomic analysis of paired cancers and adjacent normal tissues. A novel strategy for pathway analysis enabled us to discover a number of abnormalities of the colorectal cancer proteome, which included an imbalance in protein abundance of the inhibitory and activating regulators in key signal pathways, a significant elevation of proteins responsible for chromatin modification, gene expression and DNA replication and damage repair, and a decreased expression of proteins responsible for core extracellular matrix architectures. Our discovery provides indispensable information to complement available genomic data towards a better understanding of cancer biology.

An object of the invention is to provide means allowing an early detection of colon adenoma and/or colon carcinoma.

It is a further object to provide means of allowing a selective and specific detection of colon adenoma and/or colon carcinoma by a non-invasive method.

It is a further object to provide a biomarker which can be used in the detection of colorectal adenoma and/or carcinoma.

Another object of the present invention is to provide a test system for detecting colorectal adenoma or carcinoma which is cost effective and can be widely used.

Moreover, the test system should be easy to handle and more convenient for the individual to be examined for colorectal adenoma and/or carcinoma.

It is a further object of the present invention to provide a screening system for determining whether a compound is effective in the treatment of colorectal adenoma and/or carcinoma.

The objects underlying the present invention are solved by the use of CAM1, CPA3, OLM4, LAD1, DPEP1, OGFR, EPHB3, PKP3, CEAM6, SERPINB5 and MUC13 proteins, and/or their derivatives thereof as a biomarker for the detection of colorectal adenoma and/or colorectal carcinoma in an individual. The detection can be carried out in vivo and in vitro. Pursuant to a preferred embodiment, the detection is carried out in vitro. The following description on CREAM6 and its derivatives is an example to disclose the present invention, which can be used for CAM1, CPA3, OLM4, LAD1, DPEP1, OGFR, EPHB3, PKP3, SERPINB5 and MUC13 proteins, and/or their derivatives.

The objects are further solved by a method for detecting colorectal adenoma and/or colorectal carcinoma comprising the steps: a) providing an isolated sample material which has been taken from an individual, b) determining the level of CEAM6 or a derivative thereof in said isolated sample material, c) comparing the determined level of CEAM6 or a derivative thereof with one or more reference values.

The objects are further solved by a method for discriminating between colorectal adenoma and colorectal carcinoma comprising the steps: a) providing an isolated sample material which has been taken from an individual, b) determining the level of CEAM6 or a derivative thereof in said isolated sample material, c) comparing the determined level of CEAM6 or a derivative thereof with one or more reference values.

The objects are also solved by a method for monitoring the development and/or the course and/or the treatment of colorectal adenoma and/or colorectal carcinoma comprising the steps: a) providing an isolated sample material which has been taken from an individual, b) determining the level of CEAM6 or a derivative thereof in said isolated sample material, c) comparing the determined level of CEAM6 or derivative thereof with one or more reference values.

In a preferred embodiment the effectiveness of a surgical or therapeutically procedure is controlled in order to decide as to whether the colorectal adenoma and/or colorectal carcinoma is completely removed. In another embodiment the therapy of a colorectal adenoma and/or colorectal cancer patient with one or more chemical substances, antibodies, antisense-RNA, radiation, e.g. X-rays or combinations thereof is controlled in order to control the effectiveness of the treatment.

The objects are solved as well by providing a test system for detecting colorectal adenoma and/or colorectal cancer in a sample of an individual comprising: a) an antibody or a receptor which binds to an epitope of CEAM6 or a derivative thereof, b) a solid support which supports said antibody or receptor, c) a reagent for detecting the binding of said epitope of CEAM6 or a derivative thereof to said antibody or receptor.

The objects are furthermore solved by the provision of an array comprising detection molecules for detecting of colorectal adenoma and/or colorectal carcinoma in an individual comprising as detection molecule: a) a nucleic acid probe immobilized to a solid support for binding to and detecting mRNA encoding CEAM6 or a derivative thereof and/or for binding to and detecting CEAM6 proteins or derivatives thereof, or b) an antibody immobilized to a solid support for binding to and detecting of an epitope of CEAM6 or a derivative thereof, or c) a receptor immobilized to a solid support for binding to and detecting of an epitope of CEAM6 or a derivative thereof, wherein preferably each different amounts of detection molecules are immobilized to the solid support to increase the accuracy of the quantification.

The nucleic acid probe is for example selected from the group consisting of single-stranded or double-stranded DNA or RNA, aptamers and combinations thereof. Aptamers are single-stranded oligonucleotides that assume a specific, sequence-dependent shape and bind to protein targets with high specificity and affinity. Aptamers are identified using the SELEX process (Tuerk C. and Gold L. (1990) Science 249: 505-510; Ellington A D and Szostak J W. (1990) Nature 346: 818-822).

The objects are furthermore solved by a method for determining whether a compound is effective in the treatment of colorectal adenoma and/or colorectal carcinoma comprising the steps: a) treating of a colorectal adenoma or colorectal carcinoma patient with a compound, b) determining the level of CEAM6 or a derivative thereof in a sample material of said patient, and c) comparing the determined level of CEAM6 or a derivative thereof with one or more reference values.

Preferred embodiments are specified in dependent claims.

According to the present invention the term “sample material” is also designated as “sample”.

Pursuant to the present invention the term “biomarker” is meant to designate a protein or protein fragment or a nucleic acid which is indicative for the incidence of the colorectal adenoma and/or colorectal carcinoma. That means the “biomarker” is used as a mean for detecting colorectal adenoma and/or colorectal carcinoma.

The term “individual” or “individuals” is meant to designate a mammal. Preferably, the mammal is a human being such as a patient.

The term “healthy individual” or “healthy individuals” is meant to designate individual(s) not diseased of colorectal adenoma and/or colorectal carcinoma. That is to say, the term “healthy individual(s)” is used only in respect of the pathological condition of colorectal adenoma and/or colorectal carcinoma and does not exclude the individual to suffer from diseases other than colorectal adenoma and/or colorectal carcinoma.

The term “derivative thereof” is meant to describe any modification on DNA, mRNA or protein level comprising e.g. the truncated gene, fragments of said gene, a mutated gene, or modified gene. The term “gene” includes nucleic acid sequences, such as DNA, RNA, mRNA or protein sequences or oligopeptide sequences or peptide sequences. The derivative can be a modification which is an result of a deletion, substitution or insertion of the gene. The gene modification can be a result of the naturally occurring gene variability. The term “naturally occurring gene variability” means modifications which are not a result of genetic engineering. The gene modification can be a result of the processing of the gene or gene product within the body and/or a degradation product. The modification on protein level can be due to enzymatic or chemical modification within the body. For example the modification can be a glycosylation or phosphorylation. Preferably, the derivative codes for or comprises at least 5 amino acids, more preferably 10 amino acids, most preferably 20 amino acids of the unmodified protein. In one embodiment the derivative codes for at least one epitope of the respective protein.

The term “epitope” is meant to designate any structural element of a protein or peptide or any proteinaceous structure allowing the specific binding of an antibody, an antibody fragment, a protein or peptide structure or a receptor.

The methods of the present invention are carried out with sample material such as a body fluid or tissue sample which already has been isolated from the human body. Subsequently the sample material can be fractionated and/or purified. It is for example possible, to store the sample material to be tested in a freezer and to carry out the methods of the present invention at an appropriate point in time after thawing the respective sample material.

It has been surprisingly discovered by the present inventors that the protein CEAM6 or a derivative thereof can be used as a biomarker for the detection of colorectal adenoma and/or carcinoma. The inventors have now surprisingly found that the level protein CEAM6 or a derivative thereof in a tissue sample and/or body fluid is elevated in individuals having colorectal adenoma and/or carcinoma. Furthermore, the protein CEAM6 level or a derivative thereof in a tissue sample and/or body fluid can be used to distinguish healthy people from people having colorectal adenoma and/or carcinoma as well as people having colorectal adenoma from people having colorectal carcinoma.

Pursuant to the present invention, sample material can be tissue, cells or a body fluid. Preferably the sample material is a body fluid such as blood, blood plasma, blood serum, bone marrow, stool, synovial fluid, lymphatic fluid, cerebrospinal fluid, sputum, urine, mother milk, sperm, exudate and mixtures thereof. In a preferred embodiment the body fluids are fractionated with antibody affinity chromatography. The CEAM6 protein is for example eluted at pH 3.0.

Preferably, the body fluid has been isolated before carrying out the methods of the present invention. The methods of the invention are preferably carried out in vitro by a technician in a laboratory.

According to a preferred embodiment of the invention, CEAM6 is measured in blood plasma or blood serum. Blood serum can be easily obtained by taking blood from an individual to be medically examined and separating the supernatant from the clotted blood.

The level of CEAM6 or a derivative thereof in the body fluid, preferably blood serum, is higher with progressive formation of colorectal adenoma. The colorectal adenoma is a benign neoplasma which may become malign. When developing colorectal cancer from benign colorectal adenoma, the level of CEAM6 or a derivative thereof in body fluids, preferably blood serum, further is elevated.

After transformation of colorectal adenoma into colorectal cancer, the pathological condition of the afflicted individual can be further exacerbated by formation of metastasis.

The present invention provides an early stage biomarker which allows to detect the neoplastic disease at an early and still benign stage, neoplastic disease at an early stage or benign stage and/or early tumor stages. The early detection enables the physician to timely remove the colorectal adenoma and to dramatically increase the chance of the individual to survive.

Moreover, the present invention allows to monitor the level of CEAM6 or a derivative thereof in a body fluid such as blood serum over an extended period of time, such as years.

The long term monitoring allows to differentiate between healthy individuals and colorectal adenoma and/or colorectal carcinoma. The level of CEAM6 or a derivative thereof can be routinely checked, for example, once or twice a year. If an increase of the level of CEAM6 or a derivative thereof is detected this can be indicative for colorectal adenoma and/or early colorectal carcinoma. A further increase of the level of CEAM6 or a derivative thereof can then be indicative for the transformation into malign colorectal carcinoma.

Moreover, the course of the disease and/or the treatment can be monitored. If the level of CEAM6 or a derivative thereof further increases, for example after removal of the colorectal adenoma, this can be indicative for exacerbation of the pathological condition.

That means, the level of CEAM6 or a derivative thereof is a valuable clinical parameter for detecting and/or monitoring of colorectal adenoma and/or colorectal carcinoma. The level of CEAM6 or a derivative thereof in body fluids is higher after incidence of colorectal adenoma. Therefore, the level of CEAM6 or a derivative thereof is an important clinical parameter to allow an early diagnosis and, consequently, an early treatment of the disease. In a preferred embodiment patients with elevated CEAM6 levels or derivatives thereof are subsequently exanimated by colonoscopy.

The method of the invention for detection of colorectal adenoma and/or colorectal carcinoma comprises the step of providing an isolated sample material which has been taken from an individual, then determining the level of CEAM6 or a derivative thereof in the isolated sample material, and finally comparing the determined level of CEAM6 or a derivative thereof with one or more reference values. In one embodiment, one or more further biomarker(s) is/are additionally detected in an isolated sample material which has been taken from an individual, the level of the biomarker(s) is/are determined and compared with one or more respective reference values.

The reference value can be calculated as the average level of CEAM6 or a derivative thereof determined in a plurality of isolated samples of healthy individuals or individuals suffering from colorectal adenoma and/or colorectal carcinoma. This reference value can be established as a range to be considered as normal meaning that the person is healthy or suffers from colorectal adenoma and/or colorectal carcinoma. A specific value within a range can then be indicative for healthy condition or the pathological condition of colorectal adenoma and/or colorectal carcinoma. This range of reference value can be established by taking a statistically relevant number of body fluid samples, such as serum samples, of healthy individuals as it is done for any other medical parameter range such as, e.g., blood sugar. Preferably, two reference values are calculated which are designated as negative control and positive control 1. The reference value of the negative control is calculated from healthy individuals and the positive control is calculated from individuals suffering from colorectal adenoma or colorectal carcinoma. More preferably, three reference values are calculated which are designated as negative control and positive control 1 and positive control 2. Positive control 1 can be calculated from individuals suffering from colorectal carcinoma and positive control 2 can be calculated from individuals suffering from colorectal adenoma.

In an another embodiment of the present invention, the reference values can be individual reference values calculated as the average level of CEAM6 or a derivative thereof determined in a plurality of isolated samples taken from the individual over a period of time.

When monitoring the level of CEAM6 or a derivative thereof over an extended period of time, such as months or years, it is possible to establish an individual average level. The CEAM6 or a derivative thereof level can be measured, for example, from the same blood serum sample when measuring blood sugar and can be used to establish an individual calibration curve allowing to specifically detect any individual increase of the level of CEAM6 or a derivative thereof.

The reference value for further biomarkers can also be calculated in the same way as described for CEAM6. The average levels of CEAM6 or further biomarkers may be the mean or median level.

In another aspect the present invention further provides a test system for detecting colorectal adenoma and/or colorectal carcinoma in an isolated sample material of an individual. The test system is based either on the specificity of an antibody or a receptor to specifically bind to an epitope or a suitable structural element of CEAM6 or a derivative thereof or a fragment of thereof. A receptor can be any structure able to bind specifically to CEAM6 or a derivative thereof. The receptor can be, for example, an antibody fragment such as an Fab or an F(ab′).sub.2 fragment or any other protein or peptide structure being able to specifically bind to CEAM6 or a derivative thereof.

The antibody, antibody fragment or receptor is bound to a solid support such as, e.g., a plastic surface or beads to allow binding and detection of CEAM6 or a derivative thereof. For example, a conventional microtiter plate can be used as a plastic surface. The detection of the binding of CEAM6 or a derivative thereof can be effected, for example, by using a secondary antibody labelled with a detectable group. The detectable group can be, for example, a radioactive isotope or an enzyme like horseradish peroxidase or alkaline phosphatase detectable by adding a suitable substrate to produce, for example, a color or a fluorescence signal.

The test system can be an immunoassay such as an enzyme-linked immunosorbentassay (ELISA) or a radio immunoassay (RIA) or luminescence immunossay (LIA). However, any other immunological test system using the specificity of antibodies or fragments of antibodies can be used such as Western blotting or immuno precipitation.

The present invention also provides an array comprising detection molecules for detecting colorectal adenoma and/or colorectal carcinoma in an individual, wherein the detection molecule can be a nucleic acid probe immobilized on a solid support for binding to and detecting of mRNA encoding CEAM6, fragments, mutations, variants or derivatives thereof, or an antibody immobilized on a solid support for binding to and detecting of an epitope of CEAM6 or a derivative thereof, or a receptor immobilized on a solid support for binding to and detecting of an epitope of CEAM6 or a derivative thereof. Preferably, the array comprises further detection molecules which are biomarkers for detecting colorectal adenoma and colorectal carcinoma.

The nucleic acid probe can be any natural occurring or synthetic oligonucleotide or chemically modified oligonucleotides, as well as cDNA, mRNA, aptamer and the like.

Alternatively, the present invention also comprises an inverse array comprising patient samples immobilized on a solid support which can be detected by the above defined detection molecules.

Preferably the array comprises detection molecules which are immobilized to a solid surface at identifiable positions.

The term “array” as used in the present invention refers to a grouping or an arrangement, without being necessarily a regular arrangement. An array comprises preferably at least 2, more preferably 5 different sets of detection molecules or patient samples. Preferably, the array of the present invention comprises at least 50 sets of detection molecules or patient samples, further preferred at least 100 sets of detection molecules or patient samples. Pursuant to another embodiment of the invention the array of the present invention comprises at least 500 sets of detection molecules or patient samples. The detection molecule can be for example a nucleic acid probe or an antibody or a receptor.

The described array can be used in a test system according to the invention. The array can be either a micro array or a macro array.

The detection molecules are immobilized to a solid surface or support or solid support surface. This array or microarray is then screened by hybridizing nucleic acid probes prepared from patient samples or by contacting the array with proteinaceous probes prepared from patient samples.

The support can be a polymeric material such as nylon or plastic or an inorganic material such as silicon, for example a silicon wafer, or ceramic. Pursuant to a preferred embodiment, glass (SiO.sub.2) is used as solid support material. The glass can be a glass slide or glass chip. Pursuant to another embodiment of the invention the glass substrate has an atomically flat surface.

For example, the array can be comprised of immobilized nucleic acid probes able to specifically bind to mRNA of CEAM6 or a derivative thereof or antibodies specifically bind to CEAM6 protein or derivatives thereof being present in a body fluid such as serum. Another preferred embodiment is to produce cDNA by reverse transcription of CEAM6 encoding mRNA or of mRNA encoding a derivative of CEAM6 and to specifically detect the amount of respective cDNA with said array. The array technology is known to the skilled person. A quantification of the measured mRNA or cDNA or proteins, respectively, can be effected by comparison of the measured values with a standard or calibration curve of known amounts of CEAM6 or a derivative thereof mRNA or cDNA or proteins.

Preferably, different amounts of detection molecules are immobilized each on the solid support to allow an accurate quantification of the level of CEAM6 or a derivative thereof.

Pursuant to another embodiment of the invention, the level of CEAM6 or a derivative thereof is determined by liquid chromatography tandem mass spectrometry (LC/MS/MS).

LC/MS/MS analysis allows to specifically detect CEAM6 or a derivative thereof via its sequence and to quantify the amount of CEAM6 or a derivative thereof very easily.

Preferably, the CEAM6 or a derivative thereof in the isolated sample is immobilized on a chip or solid support with an activated surface. The activated surface comprises preferably immobilized antibodies against anti-CEAM6 or a derivative thereof such as, for example, rabbit polyclonal-antibodies. After binding of the CEAM6 or a derivative thereof to the antibodies, the bounded CEAM6 was digested by trypsin or other proteinases followed by a LC/MS/MS analysis in a mass spectrometer, which delivers intensity signals for determination of the CEAM6 or a derivative thereof level.

Moreover, LC/MS/MS allows to simultaneously detect other proteins which can have a relevance with respect to the detection of colorectal adenoma and/or colorectal cancer.

In an embodiment of the present invention the sensitivity and/or specificity of the detection of colorectal adenoma and/or colorectal carcinoma is enhanced by additionally detection of a further biomarker. In particular, in one embodiment the sensitivity and/or specificity of the detection of colorectal adenoma and/or colorectal carcinoma is enhanced by detection of another protein or nucleic acid in combination with CEAM6 or a derivative thereof.

Preferably, the sensitivity and specificity of the methods, arrays, test systems and uses according to the present invention are increased by the combination of detecting CEAM6 and derivatives thereof with SerpinB5 and derivatives thereof.

In a further embodiment of the present invention the sensitivity and/or specificity of the detection of colorectal adenoma and/or colorectal carcinoma is enhanced by additionally detection of MUC13, OLM4, LAD1, DPEP1, OGFR, EPHB3, PKP3, CAM1, and CPA3, or derivatives thereof in combination with CEAM6 or a derivative thereof.

The methods of the present invention can be carried out in combination with other diagnostic methods for detection of colorectal adenoma and/or colorectal carcinoma to increase the overall sensitivity and/or specificity. The detection of CEAM6 allows a very early detection of colorectal adenoma and can therefore be used as a very early marker.

Preferably, the methods of the present invention are carried out as an early detection and/or monitoring method. If the results of the methods of the present invention should indicate the incidence of colorectal adenoma and/or colorectal adenoma, further examinations such as colonoscopy should be carried out.

### BRIEF DESCRIPTION OF THE DRAWINGS

FIG. 1A Comparison of the average of protein abundance (ppm) of 11 proteins panel biomarkers and known CEA biomarker among 22 ATs, 12 TNM I&II tumors, and 10 TNM III&IV tumors. The average for adjacent tissue (AT) based on 22 AT samples, for tumors based on 12 TNM I&II tumors and 10 TNM III&IV tumors respectively.

FIG. 1B Immunohistochemistry showing differential expression of the panel of 11 proteins and CEA in CRCs and ATs. Magnification of all images was ×200.

FIG. 2A. The average abundance (ppm) of each subunit for four “housekeeping” protein complexes were based on 22 CRC samples, 22 AT samples, 94 TCGA CRC samples, 12 TCGA breast cancer samples and 3 mesenchymal stem cell (MSC) samples. Three proteome profiles from three lots of MSC supplied by RoosterBio were generated according to the same method described in Extended Methods. The ppm for each subunit is presented for the Arp⅔ complex (8 subunits).

FIG. 2B. The ppm for each subunit is presented for the Proteasome (17 subunits).

FIG. 2C. The ppm for each subunit is presented for the COP9 signalosome (9 subunits).

FIG. 2D. The ppm for each subunit is presented for the 17 TCA enzymes.

FIG. 2E. The Beck's copy number for each subunit is presented for the Arp⅔ complex (8 subunits). The Beck's copy numbers were obtained from the article published by Beck et al.

FIG. 2F. The Beck's copy number for each subunit is presented for the Proteasome (17 subunits).

FIG. 2G. The Beck's copy number for each subunit is presented for the COP9 signalosome (9 subunits).

FIG. 2H. The Beck's copy number for each subunit is presented for the 17 TCA enzymes.

FIG. 2I. Comparison of the dynamic range of abundances for four complexes.

FIG. 3A. Comparison of the proteins abundances (mass) of analyzed pathways or cellular processes between CRC and AT. For each pathway or cellular process, the total abundances was the sum of all the members' average protein abundance (ppm) which was based on 22 CRCs or 22 ATs, and the percentage was determined through dividing the summed abundances by the entire proteome protein mass 1,000,000 ppm.

FIG. 3B. Comparison of the proteins abundances (mass) of analyzed pathways or cellular processes between CRC and AT.

FIG. 4. Western blot analysis of differential expression of biomarkers in CRC and AT. For Western blot analysis equal amount of samples from paired CRC and AT were resolved by 4-12% LDS-NuPAGE gels, transferred to nitrocellulose membranes, and analyzed by western blot (WB) with corresponding antibody (SerpinB5 antibody, Product #9117, Cell Signaling) using enhanced chemiluminescence (ECL; Amersham, Piscataway, NJ).

### DETAILED DESCRIPTION OF THE PREFERED EMBODIMENTS

Generating High-Quality Proteomic Profiles

To characterize the human CRC proteome and quantify its changes, paired CRC and adjacent normal tissue (AT) samples from 22 cases of CRC patients (Table 1) were analyzed by a standardized mass spectrometry-based proteomics workflow. 44 proteomic profiles from 704 two hour LC-MS/MS runs (44 samples×16 runs) were generated. To ensure reproducibility and relative completeness, the 44 proteomic profiles were evaluated by ten groups of well-known “housekeeping” protein complexes and scored at an average of 92 out of 100. The relative abundance of identified proteins was determined based on normalized spectral abundance factors (NSAF). To quantitatively describe the relative abundance, part per million (ppm) was used as the abundance unit and a total value of 1,000,000 ppm was assigned to the proteome of each sample. Thus, the ppm value for each identified protein was calculated based on its NSAF, and the average abundance or ppm of each identified protein in CRC and AT was obtained based on 22 CRC samples and 22 AT samples, respectively.

TABLE 1

| Hospital | Biopsy | | | | | | | Pathologic |
|---|---|---|---|---|---|---|---|---|
| No | no. | Tissue | Hospital | Age | Gender | Diagnosis | Location | stage |
| 1714625 | S1-CRC- | CRC | CNM | 68 | f | rectal adenocarcinoma | rectum | T2N0M0 |
| | NN | | | | | | | |
| 1714625 | S1-AT-NN | AT | CNM | 68 | f | rectal adenocarcinoma | rectum | |
| 1695292 | S2-CRC- | CRC | CNM | 67 | f | colon adenocarcinoma | sigmoid colon | T4N1M0 |
| | NP | | | | | | | |
| 1695292 | S2-AT-NP | AT | CNM | 67 | f | colon adenocarcinoma | sigmoid colon | |
| 1713771 | S3-CRC- | CRC | CNM | 49 | m | rectal adenocarcinoma | rectum | T2N0M0 |
| | NN | | | | | | | |
| 1713771 | S3-AT-NN | AT | CNM | 49 | m | rectal adenocarcinoma | rectum | |
| 1700080 | S4-CRC- | CRC | CNM | 79 | f | colon adenocarcinoma | ascending | T4N1M0 |
| | NP | | | | | | colon | |
| 1700080 | S4-AT-NP | AT | CNM | 79 | f | colon adenocarcinoma | ascending | |
| | | | | | | | colon | |
| 1556888 | S5-CRC- | CRC | CNM | 66 | m | rectal adenocarcinoma | rectum | T4N1M0 |
| | NP | | | | | | | |
| 1556888 | S5-AT-NP | AT | CNM | 66 | m | rectal adenocarcinoma | rectum | |
| 1487028 | S6-CRC- | CRC | CNM | 57 | f | rectal adenocarcinoma | rectum | T2N0M0 |
| | NN | | | | | | | |
| 1487028 | S6-AT-NN | AT | CNM | 57 | f | rectal adenocarcinoma | rectum | |
| 755353 | S7-CRC- | CRC | CNM | 62 | m | colon adenocarcinoma | descending | T3N0M0 |
| | NN | | | | | | colon | |
| 755353 | S7-AT-NN | AT | CNM | 62 | m | colon adenocarcinoma | descending | |
| | | | | | | | colon | |
| 1067718 | S8-CRC- | CRC | CNM | 58 | f | colon adenocarcinoma | sigmoid colon | T4N0M0 |
| | NN | | | | | | | |
| 1067718 | S8-AT-NN | AT | CNM | 58 | f | colon adenocarcinoma | sigmoid colon | |
| 1078127 | S9-CRC- | CRC | CNM | 72 | m | rectal adenocarcinoma | rectum | T2N0M0 |
| | NN | | | | | | | |
| 1078127 | S9-AT-NN | AT | CNM | 72 | m | rectal adenocarcinoma | rectum | |
| 1197932 | S10-CRC- | CRC | CNM | 77 | m | rectal adenocarcinoma | rectum | T3N2M0 |
| | NP | | | | | | | |
| 1197932 | S10-AT- | AT | CNM | 77 | m | rectal adenocarcinoma | rectum | |
| | NP | | | | | | | |
| 1226388 | S11-CRC- | CRC | CNM | 42 | f | rectal adenocarcinoma | rectum | T3N0M0 |
| | NN | | | | | | | |
| 1226388 | S11-AT- | AT | CNM | 42 | f | rectal adenocarcinoma | rectum | |
| | NN | | | | | | | |
| 1230278 | S12-CRC- | CRC | CNM | 73 | m | rectal adenocarcinoma | rectum | T2N0M0 |
| | NN | | | | | | | |
| 1230278 | S12-AT- | AT | CNM | 73 | m | rectal adenocarcinoma | rectum | |
| | NN | | | | | | | |
| 1215329 | S13-CRC- | CRC | CNM | 51 | f | rectal adenocarcinoma | rectum | T3N2M0 |
| | NP | | | | | | | |
| 1215329 | S13-AT- | AT | CNM | 51 | f | rectal adenocarcinoma | rectum | |
| | NP | | | | | | | |
| 482878 | S14-CRC- | CRC | CNM | 65 | m | colon adenocarcinoma | transverse | T1N0M0 |
| | NN | | | | | | colon | |
| 482878 | S14-AT- | AT | CNM | 65 | m | colon adenocarcinoma | transverse | |
| | NN | | | | | | colon | |
| 1249727 | S15-CRC- | CRC | CNM | 83 | f | colon adenocarcinoma | sigmoid colon | T3N2M0 |
| | NP | | | | | | | |
| 1249727 | S15-AT- | AT | CNM | 83 | f | colon adenocarcinoma | sigmoid colon | |
| | NP | | | | | | | |
| 1167483 | S16-CRC- | CRC | CNM | 74 | f | rectal adenocarcinoma | rectum | T2N0M0 |
| | NN | | | | | | | |
| 1167483 | S16-AT- | AT | CNM | 74 | f | rectal adenocarcinoma | rectum | |
| | NN | | | | | | | |
| 1118276 | S17-CRC- | CRC | CNM | 56 | f | colon adenocarcinoma | sigmoid colon | T3N0M0 |
| | NN | | | | | | | |
| 1118276 | S17-AT- | AT | CNM | 56 | f | colon adenocarcinoma | sigmoid colon | |
| | NN | | | | | | | |
| 575704 | S18-CRC- | CRC | CNM | 61 | f | colon adenocarcinoma | ascending | T4N1M0 |
| | NP | | | | | | colon | |
| 575704 | S18-AT- | AT | CNM | 61 | f | colon adenocarcinoma | ascending | |
| | NP | | | | | | colon | |
| 548702 | S19-CRC- | CRC | CNM | 54 | m | colon adenocarcinoma | sigmoid colon | T3N0M0 |
| | NN | | | | | | | |
| 548702 | S19-AT- | AT | CNM | 54 | m | colon adenocarcinoma | sigmoid colon | |
| | NN | | | | | | | |
| 552625 | S20-CRC- | CRC | CNM | 65 | f | colon adenocarcinoma | transverse | T4N1M0 |
| | NP | | | | | | colon | |
| 552625 | S20-AT- | AT | CNM | 65 | f | colon adenocarcinoma | transverse | |
| | NP | | | | | | colon | |
| 1254899 | S21-CRC- | CRC | CNM | 78 | m | rectal adenocarcinoma | rectum | T3N1M0 |
| | NP | | | | | | | |
| 1254899 | S21-AT- | AT | CNM | 78 | m | rectal adenocarcinoma | rectum | |
| | NP | | | | | | | |
| 675482 | S22-CRC- | CRC | CNM | 50 | f | colon adenocarcinoma | sigmoid colon | T3N2M0 |
| | NP | | | | | | | |
| 675482 | S22-AT- | AT | CNM | 50 | f | colon adenocarcinoma | sigmoid colon | |
| | NP | | | | | | | |

At the time of writing, UniProtKB/Swiss-Prot had manually reviewed protein evidence for 20193 human genes. We identified 12380 proteins across 44 samples, which accounted for approximately 60% of all the annotated proteins in the human genome. Among them, 8832 proteins were detected in both CRCs and ATs; 10030 proteins were detected in ATs, including 1197 (9.7%) proteins undetectable in CRCs; and 11183 proteins were detected in CRCs, including 2350 (19%) proteins undetectable in ATs.

We next analyzed the distribution of the identified proteins using an Excel histogram function and revealed a normal distribution with a major peak and a minor peak. The major peak represented 62% and 60% of identified proteins with relative abundances greater than 1 ppm for CRC and AT respectively. Within this population 95% of proteins have ppm values in the range from 1 to 10000 ppm.

The minor peak represented 38% and 40% of identified proteins with relative abundance less than 1 ppm for CRC and AT respectively. The majority of proteins in the minor peak were identified by one or few peptide spectrum matches across 44 samples. Since the least abundant protein population also displayed a normal distribution, it indicated that their relative abundance could be used for a comparison between CRC and AT if it's pValue was significant (e.g. p<0.01).

CRC Proteome Landscapes

In consistency with a 60% total coverage of the human proteome, chromosomes were also evenly covered at an average of 60% with notable exceptions of the Y chromosome (18.6%) and mitochondria chromosome (85.7%) by 12380 proteins identified in CRC and AT. The high incidence for identification of the mitochondria proteins was apparently correlated with their high abundances (>10 ppm) and the low incidence for Y chromosomes was also correlated with their relatively low abundances. Although there was no apparent difference between CRC and AT for the chromosome coverage, the summed chromosome protein abundances were varied. For chromosomes 13 and 20 the summed protein abundances of CRC was 27% more than that of AT, whereas the total protein abundances for chromosomes 4, 14, 16, and the mitochondrial chromosome was 10% less in CRC than that in AT.

We next assessed the coverage according to three protein classifications as described by UniprotKB: the molecular functional classification having 14420 annotated proteins, the cellular component classification having 17465 annotated proteins, and the biological process classification having 16149 annotated proteins. The average coverage for all different classes was 67% but for each individual class the coverage varied from 40% to 90%. For known low abundance protein classes the coverage was less than 45%, while for high abundance protein classes it was more than 70%, even up to 90%. The coverage for signal transducers, receptors, nucleic acid binding transcription factors and chemoattractants was less than 40% since these proteins were least abundant. The coverage for CRC and AT showed no apparent difference. Interestingly, the summed protein abundances for protein binding transcription factors, nucleic acid binding transcription factors, and translation regulators were significantly increased while those for collagen trimers, extracellular matrix parts and extracellular matrices were decreased in CRC. These changes may reflect the fact that cancer cells were in a fast growing status with a less stable structural architectures.

Proteomic Signature of CRC

Our quantitative proteomic analysis of 22 paired CRCs and ATs identified 740 significantly differentially expressed proteins (e.g. fold change >4, p<0.01) (Table 2). Among them 613 proteins had increased expression in all 22 cases of CRC patients (p<0.01), while 127 proteins showed decreased expression (p<0.01). Interestingly, although these 740 proteins encompassed about 6% of the total proteins identified, their mass was only 1.6% and 2.5% of the total mass in the CRCs and ATs, respectively. Most of the 127 proteins decreased in CRC but enriched in AT were high-abundant proteins, which were involved in cellular architectures, metabolisms and colorectal functions. In contrast, most of the 613 proteins enriched in CRC were low-abundant proteins, which were mostly involved in the regulation of cellular processes. This explained why the total mass of the 740 proteins was 58% more in AT than that in CRC.

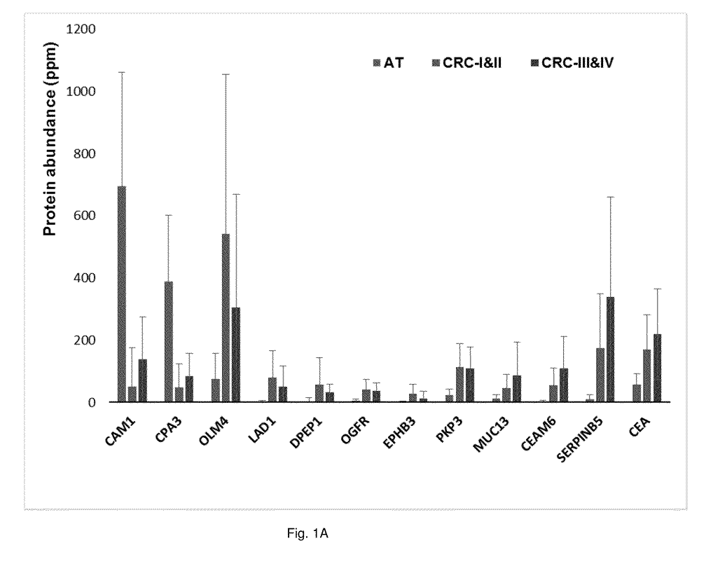

Considering the practical reality, a small panel of protein biomarkers would have more advantages. We identified a panel of 11 proteins based on the relative abundance (mean abundance in CRC >20 ppm) from the ranked 740 proteins to distinguish cancer tissues from normal colorectal tissues obviously (FIG. 1). Two enzymes, mast cell carboxypeptidase A and chymase, secreted by mast cells, were used as two positive markers for normal colorectal tissue because they were significantly decreased in CRC. Nine proteins, significantly overexpressed in cancer tissues, were used in determination of CRC. The abundance change of the 11 proteins was confirmed by immunohistochemistry assay (FIG. 1b). Among this panel, eight proteins were also reported by other studies as potential CRC signatures (discussed above), and the other three proteins (opioid growth factor receptor, Chymase, and Ladinin-1) have not been reported so far.

| | | | | | |
|---|---|---|---|---|---|
| O00762 | UBE2C | 12.31 | 0.00 | ∞ | 0.00258383 |
| O43719 | HTSF1 | 10.75 | 0.00 | ∞ | 0.003871564 |
| Q9Y4E1 | FA21C | 8.53 | 0.00 | ∞ | 0.000635828 |
| Q9HCJ6 | VAT1L | 7.45 | 0.00 | ∞ | 0.005054668 |
| Q8TDI0 | CHD5 | 7.07 | 0.00 | ∞ | 0.001640338 |
| P15428 | PGDH | 6.57 | 0.00 | ∞ | 0.008108922 |
| O94851 | MICA2 | 6.48 | 0.00 | ∞ | 0.002496059 |
| Q9GZL7 | WDR12 | 6.08 | 0.00 | ∞ | 0.005882844 |
| Q8N4B1 | SESQ1 | 5.84 | 0.00 | ∞ | 0.005592541 |
| Q9BQG0 | MBB1A | 5.55 | 0.00 | ∞ | 0.000732919 |
| O76021 | RL1D1 | 5.49 | 0.00 | ∞ | 0.004262104 |
| Q9Y2W2 | WBP11 | 4.99 | 0.00 | ∞ | 0.00590341 |
| P41743 | KPCI | 4.95 | 0.00 | ∞ | 0.001127193 |
| Q96CG8 | CTHR1 | 4.95 | 0.00 | ∞ | 0.008463863 |
| Q9BUV8 | CT024 | 4.83 | 0.00 | ∞ | 0.002749521 |
| Q15050 | RRS1 | 4.58 | 0.00 | ∞ | 0.00015297 |
| Q00613 | HSF1 | 4.53 | 0.00 | ∞ | 0.003945981 |
| Q16763 | UBE2S | 4.46 | 0.00 | ∞ | 0.001211544 |
| Q9BRJ6 | CG050 | 4.42 | 0.00 | ∞ | 0.008026981 |
| Q96SL4 | GPX7 | 4.31 | 0.00 | ∞ | 0.006063738 |
| Q9P1Y5 | CAMP3 | 3.95 | 0.00 | ∞ | 0.005218738 |
| O95639 | CPSF4 | 3.94 | 0.00 | ∞ | 0.001657374 |
| P05413 | FABPH | 3.53 | 0.00 | ∞ | 0.007359391 |
| Q9Y312 | AAR2 | 3.51 | 0.00 | ∞ | 0.000976094 |
| O75179 | ANR17 | 3.43 | 0.00 | ∞ | 0.006174241 |
| Q9BT09 | CNPY3 | 3.38 | 0.00 | ∞ | 0.001077029 |
| Q9NVP1 | DDX18 | 3.34 | 0.00 | ∞ | 0.00279031 |
| Q9H6X2 | ANTR1 | 3.34 | 0.00 | ∞ | 0.005534054 |
| Q96SY0 | VWA9 | 3.30 | 0.00 | ∞ | 0.001750168 |
| P04818 | TYSY | 3.14 | 0.00 | ∞ | 0.007953421 |
| Q8TEX9 | IPO4 | 3.13 | 0.00 | ∞ | 0.004506387 |
| Q6RFH5 | WDR74 | 2.99 | 0.00 | ∞ | 0.001121526 |
| Q9H2D1 | MFTC | 2.60 | 0.00 | ∞ | 0.008125649 |
| Q9BYC5 | FUT8 | 2.36 | 0.00 | ∞ | 0.001639542 |
| A0JNW5 | UH1BL | 2.35 | 0.00 | ∞ | 0.004905714 |
| Q9BXY0 | MAK16 | 2.34 | 0.00 | ∞ | 0.003521159 |
| Q9Y2R4 | DDX52 | 2.33 | 0.00 | ∞ | 0.00051316 |
| Q5TAP6 | UT14C | 2.21 | 0.00 | ∞ | 0.001837073 |
| P00750 | TPA | 1.92 | 0.00 | ∞ | 0.006858728 |
| Q8NFT2 | STEA2 | 1.92 | 0.00 | ∞ | 0.00290555 |
| Q9UK58 | CCNL1 | 1.90 | 0.00 | ∞ | 0.004469032 |
| Q9NYT0 | PLEK2 | 1.88 | 0.00 | ∞ | 0.003385721 |
| Q8NCL4 | GALT6 | 1.86 | 0.00 | ∞ | 0.00657232 |
| Q96CT7 | CC124 | 1.83 | 0.00 | ∞ | 0.005805859 |
| Q9UJX3 | APC7 | 1.81 | 0.00 | ∞ | 0.003555599 |
| P09936 | UCHL1 | 1.76 | 0.00 | ∞ | 0.008033758 |
| Q96KC8 | DNJC1 | 1.71 | 0.00 | ∞ | 0.008468974 |
| Q9UBD5 | ORC3 | 1.55 | 0.00 | ∞ | 0.004100912 |
| Q9NPD8 | UBE2T | 1.55 | 0.00 | ∞ | 0.003966642 |
| Q8IVS2 | FABD | 1.42 | 0.00 | ∞ | 0.005055545 |
| Q15542 | TAF5 | 1.36 | 0.00 | ∞ | 0.00350335 |
| P14672 | GTR4 | 1.35 | 0.00 | ∞ | 0.005798505 |
| Q9UHR5 | S30BP | 1.34 | 0.00 | ∞ | 0.007384901 |
| O60678 | ANM3 | 1.32 | 0.00 | ∞ | 0.001910262 |
| O60244 | MED14 | 1.28 | 0.00 | ∞ | 0.002572885 |
| P98175 | RBM10 | 1.14 | 0.00 | ∞ | 0.004931425 |
| Q16254 | E2F4 | 1.13 | 0.00 | ∞ | 0.00343212 |
| Q92917 | GPKOW | 1.12 | 0.00 | ∞ | 0.001994247 |
| Q9NVR2 | INT10 | 1.03 | 0.00 | ∞ | 0.005046354 |
| Q92609 | TBCD5 | 0.86 | 0.00 | ∞ | 0.004062223 |
| Q7Z7N9 | T179B | 0.81 | 0.00 | ∞ | 0.009523875 |
| Q14690 | RRP5 | 0.72 | 0.00 | ∞ | 0.001509908 |
| Q9NNW5 | WDR6 | 12.84 | 0.16 | 82.29 | 4.86197E−05 |
| O75718 | CRTAP | 12.44 | 0.18 | 67.58 | 0.001318332 |
| Q02388 | CO7A1 | 8.12 | 0.13 | 62.89 | 0.000211436 |
| O95832 | CLD1 | 27.45 | 0.45 | 60.61 | 1.03191E−05 |
| P18858 | DNLI1 | 5.09 | 0.09 | 58.19 | 0.004598053 |
| Q14517 | FAT1 | 2.05 | 0.04 | 55.61 | 0.003196026 |
| Q9BVP2 | GNL3 | 21.64 | 0.40 | 54.23 | 2.02513E−05 |
| Q9Y4C8 | RBM19 | 7.01 | 0.14 | 50.90 | 0.00443555 |
| Q9Y5Q8 | TF3C5 | 2.88 | 0.06 | 50.30 | 0.000357053 |
| Q9H720 | PG2IP | 5.87 | 0.12 | 49.20 | 0.001185253 |
| O00767 | ACOD | 12.57 | 0.27 | 47.22 | 0.002061649 |
| Q9NPF5 | DMAP1 | 5.78 | 0.12 | 46.58 | 0.003645621 |
| Q9HAV4 | XPO5 | 15.14 | 0.33 | 46.33 | 9.7252E−05 |
| O00411 | RPOM | 23.65 | 0.54 | 44.04 | 0.008606331 |
| O15347 | HMGB3 | 18.15 | 0.41 | 43.78 | 0.002087805 |
| Q9Y2W1 | TR150 | 4.17 | 0.10 | 42.74 | 0.005522295 |
| O15027 | SC16A | 7.03 | 0.17 | 42.59 | 0.002713857 |
| O43709 | WBS22 | 2.62 | 0.07 | 39.11 | 0.000705583 |
| Q9NU38 | PGM51 | 67.32 | 1.76 | 38.20 | 0.000279902 |
| Q8WWI1 | LMO7 | 42.36 | 1.12 | 37.79 | 0.000280061 |
| Q13895 | BYST | 5.46 | 0.15 | 37.15 | 0.00748044 |
| P35269 | T2FA | 9.79 | 0.26 | 36.99 | 0.001007463 |
| Q9Y221 | NIP7 | 2.28 | 0.06 | 36.89 | 0.002289308 |
| Q8TED0 | UTP15 | 10.23 | 0.28 | 36.58 | 0.000330974 |
| Q8WU90 | ZC3HF | 16.38 | 0.47 | 34.84 | 9.26453E−06 |
| Q16650 | TBR1 | 12.44 | 0.36 | 34.51 | 7.13659E−05 |
| P09486 | SPRC | 19.52 | 0.58 | 33.47 | 0.005683738 |
| Q8NEJ9 | NGDN | 82.03 | 2.55 | 32.21 | 0.000110307 |
| Q9BU14 | RPC3 | 6.36 | 0.20 | 32.02 | 0.00015183 |
| Q92667 | AKAP1 | 10.26 | 0.33 | 31.46 | 0.007384188 |
| Q8WWM7 | ATX2L | 3.50 | 0.11 | 31.15 | 0.00619708 |
| Q14244 | MAP7 | 5.63 | 0.19 | 30.42 | 0.003932041 |
| O94888 | UBXN7 | 22.59 | 0.76 | 29.73 | 0.000306393 |
| Q53GL7 | PAR10 | 3.59 | 0.12 | 28.75 | 0.00224657 |
| Q6UWY5 | OLFL1 | 3.15 | 0.11 | 28.17 | 0.000819738 |
| P40199 | CEAM6 | 79.65 | 2.88 | 27.69 | 9.38441E−05 |
| Q9Y3A4 | RRP7A | 14.33 | 0.53 | 27.17 | 0.005027013 |
| Q04725 | TLE2 | 17.72 | 0.65 | 27.12 | 4.24128E−06 |
| Q96GM5 | SMRD1 | 5.01 | 0.19 | 26.60 | 0.002696232 |
| P36952 | SPB5 | 249.29 | 9.85 | 25.30 | 9.11184E−05 |
| P36222 | CH3L1 | 10.79 | 0.43 | 25.22 | 0.006291687 |
| Q96HP0 | DOCK6 | 14.44 | 0.61 | 23.75 | 0.000347101 |
| Q14671 | PUM1 | 23.83 | 1.06 | 22.57 | 2.63203E−05 |
| P55081 | MFAP1 | 3.12 | 0.14 | 21.73 | 0.00333933 |
| Q5TC82 | RC3H1 | 4.00 | 0.19 | 21.21 | 0.008278356 |
| Q99575 | POP1 | 8.39 | 0.40 | 21.19 | 0.00339709 |
| O75298 | RTN2 | 44.94 | 2.13 | 21.13 | 1.96903E−06 |
| Q9NU22 | MDN1 | 0.78 | 0.04 | 20.37 | 0.005299225 |
| Q8IVL6 | P3H3 | 7.41 | 0.37 | 20.18 | 0.00692006 |
| Q53FP2 | TMM35 | 27.26 | 1.36 | 20.11 | 9.50426E−05 |
| O43290 | SNUT1 | 11.50 | 0.57 | 20.10 | 0.001189978 |
| Q9Y520 | PRC2C | 3.06 | 0.15 | 19.83 | 0.000741181 |
| Q96DX5 | ASB9 | 5.50 | 0.28 | 19.75 | 0.009848765 |
| Q96EY4 | TMA16 | 3.96 | 0.20 | 19.73 | 0.005983501 |
| O14562 | UBFD1 | 9.20 | 0.47 | 19.62 | 0.000204944 |
| Q99808 | S29A1 | 33.73 | 1.73 | 19.54 | 6.11283E−06 |
| Q9BVJ6 | UT14A | 5.31 | 0.28 | 19.31 | 0.001187399 |
| Q8WUF5 | IASPP | 17.62 | 0.96 | 18.38 | 0.000135955 |
| Q13442 | HAP28 | 3.82 | 0.21 | 18.05 | 0.001326412 |
| Q96MG7 | MAGG1 | 1.11 | 0.06 | 17.74 | 0.003918852 |
| Q6P9B9 | INT5 | 14.13 | 0.80 | 17.74 | 0.007714491 |
| O43761 | SNG3 | 19.54 | 1.10 | 17.69 | 1.94589E−05 |
| Q9UJA5 | TRM6 | 13.29 | 0.76 | 17.47 | 0.000165477 |
| Q6P1Q9 | MET2B | 2.91 | 0.17 | 17.43 | 0.004123371 |
| Q8WXD5 | GEMI6 | 7.64 | 0.44 | 17.28 | 8.84042E−05 |
| Q9UHR4 | BI2L1 | 3.15 | 0.18 | 17.11 | 0.001025199 |
| Q92805 | GOGA1 | 5.04 | 0.30 | 16.94 | 0.000719334 |
| Q6UN15 | FIP1 | 5.92 | 0.35 | 16.70 | 0.008218913 |
| Q5VTR2 | BRE1A | 6.88 | 0.41 | 16.63 | 0.000346305 |
| Q9UH62 | ARMX3 | 3.98 | 0.24 | 16.61 | 0.007853336 |
| Q9Y653 | GPR56 | 2.28 | 0.14 | 16.52 | 0.009537837 |
| Q969X6 | CIR1A | 21.88 | 1.33 | 16.44 | 4.97636E−06 |
| P82094 | TMF1 | 24.75 | 1.52 | 16.27 | 0.009485675 |
| Q9GZR7 | DDX24 | 4.49 | 0.28 | 16.06 | 0.00187829 |
| Q9H9C1 | SPE39 | 10.52 | 0.66 | 16.01 | 3.20881E−05 |
| Q8N9N8 | EIF1A | 2.01 | 0.13 | 15.74 | 0.00492444 |
| A5PLL7 | TM189 | 8.00 | 0.51 | 15.71 | 0.002982846 |
| O76070 | SYUG | 2.45 | 0.16 | 15.68 | 0.009611137 |
| Q9NWH9 | SLTM | 2.85 | 0.18 | 15.52 | 0.00050321 |
| Q8WYP5 | ELYS | 1.19 | 0.08 | 15.35 | 0.004556264 |
| Q9BTE3 | MCMBP | 8.29 | 0.54 | 15.33 | 0.005293237 |
| P17213 | BPI | 3.37 | 0.22 | 15.30 | 0.003518762 |
| Q9H6S3 | ES8L2 | 4.25 | 0.28 | 15.11 | 0.001682913 |
| Q96AE7 | TTC17 | 27.65 | 1.84 | 15.07 | 2.5928E−06 |
| Q5SY16 | NOL9 | 21.00 | 1.40 | 15.05 | 0.009215468 |
| O76041 | NEBL | 2.80 | 0.19 | 14.91 | 0.000674282 |
| P15529 | MCP | 38.66 | 2.60 | 14.85 | 9.14294E−07 |
| Q9H8H0 | NOL11 | 15.32 | 1.04 | 14.71 | 0.000487307 |
| Q8N126 | CADM3 | 15.27 | 1.05 | 14.51 | 0.007837616 |
| Q9H583 | HEAT1 | 19.14 | 1.34 | 14.32 | 1.38438E−05 |
| O94875 | SRBS2 | 8.49 | 0.60 | 14.18 | 0.001375247 |
| P16444 | DPEP1 | 45.90 | 3.25 | 14.14 | 0.004880986 |
| Q9H2C0 | GAN | 18.03 | 1.28 | 14.06 | 9.02959E−05 |
| Q5MNZ6 | WIPI3 | 3.47 | 0.25 | 13.80 | 0.009820537 |
| Q9Y2S0 | RPAC2 | 13.01 | 0.95 | 13.74 | 0.000634602 |
| Q5VW38 | GP107 | 10.15 | 0.74 | 13.69 | 0.000144814 |
| Q15061 | WDR43 | 17.01 | 1.24 | 13.67 | 2.13124E−07 |
| Q9Y6N7 | ROBO1 | 3.32 | 0.24 | 13.59 | 0.006848063 |
| Q9Y5J1 | UTP18 | 11.18 | 0.83 | 13.51 | 0.000158075 |
| Q9H6T3 | RPAP3 | 2.20 | 0.16 | 13.50 | 0.009950482 |
| Q8IXQ6 | PARP9 | 8.56 | 0.65 | 13.25 | 0.002355883 |
| Q9BVI4 | NOC4L | 11.00 | 0.84 | 13.11 | 0.008208622 |
| P18615 | NELFE | 5.90 | 0.45 | 13.01 | 0.010365816 |
| Q6ZRP7 | QSOX2 | 3.38 | 0.26 | 12.98 | 0.00725217 |
| O14646 | CHD1 | 3.44 | 0.27 | 12.98 | 0.005190953 |
| Q8N8A6 | DDX51 | 2.21 | 0.17 | 12.97 | 0.010373285 |
| Q9NYF8 | BCLF1 | 0.67 | 0.05 | 12.85 | 0.001628623 |
| O95214 | LERL1 | 6.89 | 0.54 | 12.74 | 0.000600231 |
| P46087 | NOP2 | 8.82 | 0.69 | 12.74 | 0.001151286 |
| Q9Y289 | SC5A6 | 15.64 | 1.23 | 12.71 | 0.000768869 |
| Q9H089 | LSG1 | 4.63 | 0.37 | 12.66 | 0.005127764 |
| Q6ZRV2 | FA83H | 2.60 | 0.21 | 12.65 | 0.007487826 |
| O00443 | P3C2A | 1.82 | 0.15 | 12.45 | 0.009231963 |
| Q13123 | RED | 3.66 | 0.29 | 12.45 | 0.001504729 |
| O43395 | PRPF3 | 14.14 | 1.14 | 12.41 | 0.002811032 |
| Q9H0H0 | INT2 | 10.01 | 0.81 | 12.38 | 0.002811211 |
| Q9HC21 | TPC | 44.48 | 3.59 | 12.38 | 0.002750438 |
| Q05519 | SRS11 | 6.22 | 0.50 | 12.37 | 0.008818649 |
| Q8N6T3 | ARFG1 | 13.72 | 1.12 | 12.20 | 6.07178E−05 |
| Q9NX61 | T161A | 4.30 | 0.35 | 12.13 | 2.98825E−05 |
| O15075 | DCLK1 | 22.49 | 1.86 | 12.12 | 0.002787862 |
| O60934 | NBN | 5.01 | 0.42 | 12.03 | 0.000770558 |
| Q9NRF9 | DPOE3 | 25.40 | 2.14 | 11.89 | 0.000397543 |
| O15327 | INP4B | 2.14 | 0.18 | 11.85 | 0.006546352 |
| Q9NUG6 | PDRG1 | 3.05 | 0.26 | 11.81 | 0.009295823 |
| P04920 | B3A2 | 2.65 | 0.22 | 11.81 | 0.001608708 |
| Q9BRP8 | WIBG | 18.63 | 1.60 | 11.65 | 0.002564229 |
| Q9NY61 | AATF | 1.18 | 0.10 | 11.61 | 0.009625929 |
| O75787 | RENR | 12.74 | 1.10 | 11.59 | 1.50685E−05 |
| Q9GZU8 | F192A | 4.39 | 0.38 | 11.50 | 0.003028146 |
| Q9BYF1 | ACE2 | 2.42 | 0.21 | 11.50 | 0.008698363 |
| P32189 | GLPK | 13.13 | 1.14 | 11.49 | 0.000619031 |
| Q7Z4I7 | LIMS2 | 14.84 | 1.32 | 11.23 | 0.000137022 |
| P0CB43 | | 4.88 | 0.44 | 11.18 | 0.001052367 |
| O15381 | NVL | 1.86 | 0.17 | 11.13 | 0.007204064 |
| Q9BQP7 | MGME1 | 8.51 | 0.77 | 11.09 | 0.000882905 |
| Q9H4L5 | OSBL3 | 9.43 | 0.85 | 11.07 | 0.006360388 |
| Q9Y446 | PKP3 | 30.99 | 2.80 | 11.06 | 2.06237E−05 |
| Q9Y3D8 | KAD6 | 10.81 | 0.98 | 10.97 | 0.001795459 |
| P54753 | EPHB3 | 21.02 | 1.92 | 10.97 | 0.003163735 |
| Q96C90 | PP14B | 56.50 | 5.20 | 10.87 | 4.44918E−06 |
| Q8TEB1 | DCA11 | 1.91 | 0.18 | 10.85 | 0.005672947 |
| Q6NZY4 | ZCHC8 | 3.72 | 0.34 | 10.83 | 0.007989038 |
| Q9H9Y6 | RPA2 | 2.37 | 0.22 | 10.83 | 0.008609369 |
| Q9P2K3 | RCOR3 | 1.37 | 0.13 | 10.78 | 0.002977496 |
| Q96HR3 | MED30 | 21.66 | 2.01 | 10.76 | 5.77288E−06 |
| P52701 | MSH6 | 10.80 | 1.00 | 10.75 | 8.33315E−05 |
| Q86UL3 | GPAT4 | 11.19 | 1.04 | 10.75 | 0.003543096 |
| O43159 | RRP8 | 3.16 | 0.29 | 10.75 | 0.003589002 |
| Q14BN4 | SLMAP | 7.19 | 0.67 | 10.69 | 0.000733511 |
| O75150 | BRE1B | 29.55 | 2.80 | 10.57 | 4.42995E−06 |
| Q9Y4K1 | AIM1 | 2.32 | 0.22 | 10.56 | 0.001055095 |
| P41214 | EIF2D | 4.29 | 0.41 | 10.48 | 0.000936532 |
| Q03701 | CEBPZ | 2.33 | 0.22 | 10.46 | 0.0010723 |
| Q70J99 | UN13D | 33.14 | 3.19 | 10.37 | 0.000103846 |
| P16220 | CREB1 | 2.44 | 0.24 | 10.34 | 0.005228367 |
| Q9UKX7 | NUP50 | 20.08 | 1.95 | 10.29 | 0.000362019 |
| O75023 | LIRB5 | 18.09 | 1.77 | 10.21 | 7.64688E−05 |
| Q8WUP2 | FBLI1 | 2.16 | 0.21 | 10.20 | 0.003937928 |
| O15066 | KIF3B | 5.33 | 0.52 | 10.18 | 0.009085686 |
| Q9UIG0 | BAZ1B | 1.52 | 0.15 | 10.13 | 0.00892706 |
| Q2NL82 | TSR1 | 8.64 | 0.85 | 10.11 | 0.010224233 |
| Q14541 | HNF4G | 27.92 | 2.76 | 10.10 | 0.000556527 |
| Q9Y3T9 | NOC2L | 30.27 | 3.02 | 10.03 | 2.18211E−06 |
| Q8IVT2 | MISP | 20.72 | 2.07 | 10.02 | 0.000910059 |
| O00469 | PLOD2 | 29.83 | 2.99 | 9.96 | 2.39119E−05 |
| P78318 | IGBP1 | 12.24 | 1.23 | 9.93 | 1.06675E−05 |
| O95810 | SDPR | 3.10 | 0.31 | 9.86 | 0.00537181 |
| Q05209 | PTN12 | 1.22 | 0.12 | 9.84 | 0.005181809 |
| P14384 | CBPM | 47.86 | 4.88 | 9.81 | 1.22294E−05 |
| Q86Y56 | HEAT2 | 7.51 | 0.77 | 9.79 | 0.000396231 |
| Q9H6E4 | CC134 | 12.48 | 1.30 | 9.63 | 0.001367134 |
| Q99848 | EBP2 | 6.56 | 0.68 | 9.62 | 0.000184118 |
| Q9UBL3 | ASH2L | 2.47 | 0.26 | 9.62 | 0.006204619 |
| Q96GQ7 | DDX27 | 5.89 | 0.61 | 9.60 | 0.007606352 |
| Q14137 | BOP1 | 3.98 | 0.42 | 9.58 | 0.004556002 |
| Q69YN4 | VIR | 19.14 | 2.01 | 9.50 | 0.001132098 |
| O43808 | PM34 | 7.04 | 0.74 | 9.48 | 0.004484995 |
| Q9H974 | QTRD1 | 2.27 | 0.24 | 9.48 | 0.006522051 |
| Q7Z2T5 | TRM1L | 14.65 | 1.55 | 9.45 | 0.001758158 |
| O00267 | SPT5H | 7.90 | 0.84 | 9.38 | 0.000691437 |
| O95453 | PARN | 5.62 | 0.60 | 9.38 | 0.003861993 |
| O15379 | HDAC3 | 4.94 | 0.53 | 9.36 | 0.001745588 |
| Q8IWB1 | IPRI | 2.38 | 0.25 | 9.36 | 0.007183767 |
| Q08752 | PPID | 7.12 | 0.76 | 9.34 | 0.003794014 |
| Q14676 | MDC1 | 1.13 | 0.12 | 9.33 | 0.00020977 |
| Q92604 | LGAT1 | 8.44 | 0.90 | 9.33 | 0.005922578 |
| Q9NZN8 | CNOT2 | 2.35 | 0.25 | 9.31 | 0.001553628 |
| Q15007 | FL2D | 47.77 | 5.18 | 9.22 | 0.000848717 |
| Q9H1D9 | RPC6 | 4.17 | 0.45 | 9.19 | 0.005506763 |
| Q32P28 | P3H1 | 40.21 | 4.39 | 9.15 | 5.3257E−07 |
| Q9Y5V0 | ZN706 | 0.69 | 0.08 | 9.12 | 0.007998814 |
| Q9P287 | BCCIP | 14.66 | 1.61 | 9.08 | 0.007821868 |
| Q9UNF1 | MAGD2 | 41.69 | 4.62 | 9.02 | 0.000367373 |
| Q8IZV5 | RDH10 | 8.50 | 0.94 | 8.99 | 0.00178712 |
| P78345 | RPP38 | 7.91 | 0.88 | 8.96 | 0.001677789 |
| Q8TEQ6 | GEMI5 | 9.54 | 1.06 | 8.96 | 9.0507E−06 |
| P35658 | NU214 | 7.88 | 0.88 | 8.93 | 0.000714214 |
| Q9Y5X2 | SNX8 | 95.97 | 10.78 | 8.90 | 5.26566E−06 |
| P07093 | GDN | 3.20 | 0.36 | 8.87 | 0.010273894 |
| Q9NRL2 | BAZ1A | 17.40 | 1.98 | 8.80 | 4.31076E−06 |
| Q9UHN6 | TMEM2 | 177.07 | 20.19 | 8.77 | 2.2451E−08 |
| Q9H2P0 | ADNP | 6.76 | 0.77 | 8.72 | 0.000408155 |
| Q9NR12 | PDLI7 | 7.06 | 0.81 | 8.68 | 0.000496265 |
| O14558 | HSPB6 | 2.38 | 0.28 | 8.65 | 0.007620853 |
| Q9Y3C1 | NOP16 | 14.46 | 1.67 | 8.64 | 7.58182E−06 |
| Q9BZE4 | NOG1 | 40.08 | 4.65 | 8.63 | 1.9303E−05 |
| Q92835 | SHIP1 | 13.24 | 1.54 | 8.59 | 0.000255201 |
| Q8N3C0 | ASCC3 | 3.49 | 0.41 | 8.55 | 0.000266866 |
| P57678 | GEMI4 | 1.94 | 0.23 | 8.51 | 0.006625587 |
| Q15392 | DHC24 | 9.32 | 1.10 | 8.50 | 0.000396254 |
| Q15643 | TRIPB | 2.70 | 0.32 | 8.48 | 0.008934012 |
| Q9BVL2 | NUPL1 | 6.43 | 0.76 | 8.47 | 6.28623E−06 |
| P49790 | NU153 | 5.09 | 0.61 | 8.38 | 0.00637484 |
| Q460N5 | PAR14 | 8.48 | 1.01 | 8.37 | 0.009380795 |
| A3KN83 | SBNO1 | 4.96 | 0.59 | 8.37 | 0.00768995 |
| Q15124 | PGM5 | 0.95 | 0.11 | 8.35 | 0.007997518 |
| Q5T280 | CI114 | 39.27 | 4.70 | 8.35 | 3.00256E−06 |
| P23921 | RIR1 | 19.55 | 2.34 | 8.34 | 8.40243E−05 |
| Q9BY77 | PDIP3 | 2.89 | 0.35 | 8.31 | 0.005739552 |
| Q9UKV3 | ACINU | 20.04 | 2.43 | 8.26 | 0.000460375 |
| Q9H9Y2 | RPF1 | 4.61 | 0.56 | 8.25 | 0.003500468 |
| P04271 | S100B | 22.49 | 2.73 | 8.22 | 0.00591513 |
| Q9BSC4 | NOL10 | 18.41 | 2.24 | 8.20 | 0.000936071 |
| P23588 | IF4B | 18.97 | 2.31 | 8.20 | 0.000150313 |
| Q9NTJ3 | SMC4 | 37.48 | 4.62 | 8.12 | 6.28744E−05 |
| Q96I25 | SPF45 | 12.08 | 1.50 | 8.05 | 0.001062959 |
| Q9H0C8 | ILKAP | 8.33 | 1.04 | 8.04 | 0.003089629 |
| O95302 | FKBP9 | 11.85 | 1.47 | 8.04 | 0.000276546 |
| Q14767 | LTBP2 | 5.80 | 0.72 | 8.01 | 0.001864555 |
| O95071 | UBR5 | 2.76 | 0.35 | 7.98 | 0.003212425 |
| Q9UQ35 | SRRM2 | 6.27 | 0.79 | 7.97 | 0.001067044 |
| O43490 | PROM1 | 6.47 | 0.82 | 7.93 | 0.005116528 |
| Q9C0E2 | XPO4 | 10.81 | 1.36 | 7.93 | 0.001643155 |
| Q8N3X1 | FNBP4 | 11.93 | 1.51 | 7.91 | 0.000294198 |
| P40222 | TXLNA | 49.12 | 6.26 | 7.85 | 2.67194E−05 |
| Q13946 | PDE7A | 4.40 | 0.57 | 7.75 | 0.006576331 |
| P24468 | COT2 | 15.27 | 1.98 | 7.70 | 9.12102E−05 |
| Q9H173 | SIL1 | 3.38 | 0.44 | 7.66 | 0.00561057 |
| Q9NQ55 | SSF1 | 2.18 | 0.29 | 7.66 | 0.006843541 |
| P17677 | NEUM | 24.77 | 3.24 | 7.65 | 0.001859165 |
| Q9H788 | SH24A | 3.61 | 0.48 | 7.59 | 0.002247015 |
| P51787 | KCNQ1 | 10.17 | 1.34 | 7.58 | 0.000897177 |
| O75594 | PGRP1 | 8.16 | 1.08 | 7.57 | 0.006490356 |
| O15530 | PDPK1 | 4.20 | 0.56 | 7.56 | 0.0045346 |
| Q9NX57 | RAB20 | 23.95 | 3.17 | 7.56 | 3.44994E−05 |
| Q6UB35 | C1TM | 31.23 | 4.15 | 7.52 | 5.39213E−05 |
| P29375 | KDM5A | 1.54 | 0.21 | 7.50 | 0.000312831 |
| O75909 | CCNK | 6.66 | 0.89 | 7.49 | 0.001600143 |
| Q9P0P0 | RN181 | 1.90 | 0.25 | 7.46 | 0.007679117 |
| A8K0Z3 | WASH1 | 7.34 | 0.99 | 7.45 | 0.001000737 |
| P28702 | RXRB | 14.47 | 1.94 | 7.44 | 0.005204153 |
| O00165 | HAX1 | 4.84 | 0.65 | 7.43 | 0.005938409 |
| Q9UN86 | G3BP2 | 9.62 | 1.30 | 7.40 | 0.001683647 |
| Q32P41 | TRM5 | 2.04 | 0.28 | 7.37 | 0.004593244 |
| P0C6E5 | HMG3M | 22.55 | 3.07 | 7.34 | 1.4669E−05 |
| O75152 | ZC11A | 11.21 | 1.53 | 7.32 | 0.006214801 |
| Q9Y450 | HBS1L | 12.29 | 1.68 | 7.32 | 0.006439851 |
| P31350 | RIR2 | 28.14 | 3.86 | 7.29 | 1.30195E−05 |
| P13984 | T2FB | 24.62 | 3.40 | 7.24 | 0.002986227 |
| Q9Y6K5 | OAS3 | 23.95 | 3.31 | 7.23 | 1.54636E−07 |
| Q15397 | K0020 | 37.64 | 5.21 | 7.23 | 3.30305E−07 |
| Q9NZN5 | ARHGC | 1.56 | 0.22 | 7.20 | 0.005870081 |
| Q96FX7 | TRM61 | 23.93 | 3.32 | 7.20 | 5.93759E−07 |
| P13674 | P4HA1 | 48.11 | 6.69 | 7.19 | 3.04699E−09 |
| O00139 | KIF2A | 2.18 | 0.30 | 7.19 | 0.004010357 |
| Q96K37 | S35E1 | 9.56 | 1.33 | 7.17 | 0.001113471 |
| P85037 | FOXK1 | 33.52 | 4.68 | 7.16 | 0.000367745 |
| Q8N1Q1 | CAH13 | 11.00 | 1.54 | 7.12 | 0.000333086 |
| Q9UKL0 | RCOR1 | 9.28 | 1.31 | 7.11 | 0.000280346 |
| P20839 | IMDH1 | 13.87 | 1.95 | 7.10 | 1.29498E−05 |
| Q16270 | IBP7 | 18.73 | 2.64 | 7.10 | 0.000173306 |
| Q15800 | MSMO1 | 28.06 | 3.96 | 7.08 | 0.000728569 |
| Q9BUL9 | RPP25 | 7.57 | 1.07 | 7.06 | 0.003109181 |
| O15357 | SHIP2 | 5.01 | 0.72 | 6.98 | 0.004892186 |
| Q8N127 | THOC2 | 13.87 | 1.99 | 6.98 | 5.99491E−05 |
| Q02809 | PLOD1 | 49.33 | 7.11 | 6.94 | 2.52997E−08 |
| Q14997 | PSME4 | 2.98 | 0.44 | 6.81 | 0.001951961 |
| P33991 | MCM4 | 45.69 | 6.75 | 6.77 | 0.000195691 |
| Q96PZ0 | PUS7 | 6.03 | 0.89 | 6.76 | 0.004626252 |
| Q70UQ0 | IKIP | 2.90 | 0.43 | 6.74 | 0.000464182 |
| Q8WUX9 | CHMP7 | 2.36 | 0.35 | 6.73 | 0.001833987 |
| P57772 | SELB | 1.89 | 0.28 | 6.71 | 0.005974007 |
| P42338 | PK3CB | 1.41 | 0.21 | 6.70 | 0.007734176 |
| O15460 | P4HA2 | 33.57 | 5.07 | 6.62 | 0.000261686 |
| Q9UNX4 | WDR3 | 12.11 | 1.83 | 6.61 | 2.11013E−05 |
| Q92791 | SC65 | 40.98 | 6.23 | 6.58 | 9.1086E−08 |
| Q9NW13 | RBM28 | 8.36 | 1.27 | 6.57 | 0.009030067 |
| O15355 | PPM1G | 9.02 | 1.38 | 6.54 | 0.009092327 |
| O00515 | LAD1 | 4.13 | 0.63 | 6.51 | 0.003142266 |
| Q9HOAO | NAT10 | 43.74 | 6.74 | 6.49 | 4.01997E−07 |
| Q14247 | SRC8 | 105.13 | 16.23 | 6.48 | 6.44711E−05 |
| Q9NWS0 | PIHD1 | 26.09 | 4.04 | 6.46 | 0.003510935 |
| Q96RS6 | NUDC1 | 4.76 | 0.74 | 6.42 | 0.006011791 |
| Q9NTN3 | S35D1 | 76.14 | 11.87 | 6.42 | 9.68599E−06 |
| Q6DKI1 | RL7L | 47.00 | 7.33 | 6.42 | 3.63767E−05 |
| Q9NP77 | SSU72 | 20.72 | 3.24 | 6.40 | 0.000719144 |
| P52292 | IMA1 | 10.65 | 1.66 | 6.40 | 0.000191878 |
| Q8TDB6 | DTX3L | 13.35 | 2.09 | 6.38 | 0.000182822 |
| Q92536 | YLAT2 | 3.05 | 0.48 | 6.37 | 0.007127054 |
| Q9UDY2 | ZO2 | 44.94 | 7.09 | 6.34 | 0.000306991 |
| P57076 | CU059 | 3.99 | 0.63 | 6.34 | 0.001019195 |
| Q92759 | TF2H4 | 3.86 | 0.61 | 6.32 | 0.008515983 |
| P78316 | NOP14 | 12.75 | 2.02 | 6.32 | 0.004544355 |
| Q96D15 | RCN3 | 7.65 | 1.21 | 6.32 | 0.00030918 |
| Q9UBP6 | TRMB | 11.14 | 1.76 | 6.32 | 0.000206614 |
| Q9UKD2 | MRT4 | 5.02 | 0.80 | 6.29 | 0.00295845 |
| Q8IVF7 | FMNL3 | 6.98 | 1.11 | 6.27 | 0.003178242 |
| Q9UHC9 | NPCL1 | 1.72 | 0.27 | 6.26 | 0.00832513 |
| Q9BQ39 | DDX50 | 23.50 | 3.76 | 6.25 | 7.8179E−06 |
| Q9UHA3 | RLP24 | 19.37 | 3.11 | 6.23 | 0.000476088 |
| P40818 | UBP8 | 6.18 | 0.99 | 6.22 | 0.001105274 |
| P20592 | MX2 | 6.50 | 1.05 | 6.21 | 0.001501061 |
| Q8WTV0 | SCRB1 | 5.11 | 0.83 | 6.19 | 0.003505575 |
| P33993 | MCM7 | 79.37 | 12.85 | 6.17 | 4.23182E−06 |
| P10645 | CMGA | 6.77 | 1.10 | 6.17 | 0.001185169 |
| Q68CQ4 | DIEXF | 3.62 | 0.59 | 6.15 | 0.007157188 |
| Q15637 | SF01 | 9.06 | 1.48 | 6.11 | 0.009626717 |
| Q15554 | TERF2 | 6.13 | 1.01 | 6.10 | 0.009885195 |
| Q86VM9 | ZCH18 | 8.49 | 1.39 | 6.09 | 0.00083076 |
| Q96G21 | IMP4 | 15.20 | 2.50 | 6.09 | 0.000735309 |
| Q13459 | MYO9B | 1.68 | 0.28 | 6.08 | 0.004471215 |
| Q63HN8 | RN213 | 31.48 | 5.18 | 6.07 | 0.000142399 |
| Q9NRX1 | PNO1 | 9.06 | 1.49 | 6.07 | 0.003151754 |
| Q9UPU7 | TBD2B | 26.55 | 4.38 | 6.06 | 4.96554E−05 |
| Q96B96 | TM159 | 7.56 | 1.25 | 6.06 | 0.009531936 |
| Q9NVX2 | NLE1 | 16.51 | 2.73 | 6.05 | 0.000796378 |
| P30260 | CDC27 | 1.57 | 0.26 | 6.04 | 0.009025974 |
| Q07817 | B2CL1 | 7.42 | 1.23 | 6.02 | 0.002612459 |
| Q0P6H9 | TMM62 | 52.24 | 8.68 | 6.02 | 1.23505E−09 |
| Q01650 | LAT1 | 6.03 | 1.01 | 5.98 | 0.001407531 |
| P51116 | FXR2 | 7.61 | 1.28 | 5.94 | 0.0004056 |
| Q8IYB3 | SRRM1 | 11.22 | 1.89 | 5.94 | 0.006129414 |
| Q9UM22 | EPDR1 | 7.58 | 1.28 | 5.90 | 0.006074719 |
| Q9C040 | TRIM2 | 32.52 | 5.52 | 5.90 | 2.38789E−06 |
| Q5T0N5 | FBP1L | 9.63 | 1.64 | 5.88 | 6.63967E−05 |
| A6NKT7 | RGPD3 | 15.11 | 2.57 | 5.87 | 3.79313E−05 |
| Q06787 | FMR1 | 11.56 | 1.97 | 5.87 | 2.36518E−05 |
| Q13395 | TARB1 | 17.47 | 2.98 | 5.87 | 8.42367E−05 |
| Q5SRE5 | NU188 | 11.08 | 1.89 | 5.85 | 0.005376425 |
| Q9BTD8 | RBM42 | 149.38 | 25.56 | 5.84 | 1.29485E−06 |
| Q13188 | STK3 | 6.28 | 1.07 | 5.84 | 0.002789832 |
| Q6UX06 | OLFM4 | 433.73 | 74.40 | 5.83 | 0.00078041 |
| Q8NFJ5 | RAI3 | 10.77 | 1.86 | 5.80 | 0.000910571 |
| Q9UG63 | ABCF2 | 3.40 | 0.59 | 5.80 | 0.002414351 |
| Q9UJX5 | APC4 | 7.80 | 1.35 | 5.80 | 0.000638103 |
| Q9BQ75 | CMS1 | 10.30 | 1.78 | 5.80 | 5.2345E−05 |
| Q9NX58 | LYAR | 8.14 | 1.41 | 5.79 | 0.00295343 |
| O43818 | U3IP2 | 12.32 | 2.13 | 5.79 | 0.007294386 |
| Q8IY47 | KBTB2 | 0.69 | 0.12 | 5.76 | 0.004449884 |
| Q86WB0 | NIPA | 39.22 | 6.83 | 5.75 | 0.001494941 |
| P0DJD0 | RGPD1 | 9.20 | 1.60 | 5.74 | 0.000356972 |
| Q8NEN9 | PDZD8 | 57.27 | 9.98 | 5.74 | 9.46208E−07 |
| P49792 | RBP2 | 24.59 | 4.29 | 5.73 | 1.14052E−05 |
| Q9UJV9 | DDX41 | 4.41 | 0.77 | 5.72 | 0.004574242 |
| O60637 | TSN3 | 1.61 | 0.28 | 5.70 | 0.006828216 |
| Q96PP9 | GBP4 | 4.25 | 0.75 | 5.67 | 0.005460999 |
| O75376 | NCOR1 | 2.14 | 0.38 | 5.67 | 0.00105323 |
| Q15785 | TOM34 | 75.70 | 13.37 | 5.66 | 1.19236E−05 |
| Q9NRN5 | OLFL3 | 16.00 | 2.83 | 5.64 | 0.009222217 |
| Q15025 | TNIP1 | 11.57 | 2.05 | 5.64 | 0.000156823 |
| Q8N3U4 | STAG2 | 11.48 | 2.04 | 5.64 | 0.000384935 |
| Q9BQ61 | CS043 | 5.93 | 1.06 | 5.62 | 0.00365111 |
| P52630 | STAT2 | 11.24 | 2.00 | 5.61 | 9.4292E−05 |
| P57081 | WDR4 | 35.11 | 6.26 | 5.61 | 3.6994E−06 |
| Q96BP2 | CHCH1 | 18.01 | 3.22 | 5.59 | 0.000110251 |
| Q9Y5Q9 | TF3C3 | 37.77 | 6.78 | 5.57 | 0.007984587 |
| Q9Y2L1 | RRP44 | 47.77 | 8.58 | 5.57 | 3.28587E−06 |
| P50443 | S26A2 | 8.94 | 1.61 | 5.56 | 0.000891143 |
| Q8IYS1 | P20D2 | 21.58 | 3.89 | 5.55 | 2.56299E−05 |
| Q9ULR3 | PPM1H | 16.11 | 2.91 | 5.53 | 0.000348732 |
| O75607 | NPM3 | 9.64 | 1.75 | 5.51 | 0.006948005 |
| Q9NRG0 | CHRC1 | 8.08 | 1.47 | 5.50 | 0.001711082 |
| P05161 | ISG15 | 64.18 | 11.71 | 5.48 | 0.001298083 |
| Q5VTL8 | PR38B | 9.38 | 1.71 | 5.48 | 4.51231E−05 |
| Q01968 | OCRL | 4.05 | 0.74 | 5.47 | 0.002516598 |
| Q8TDN6 | BRX1 | 3.97 | 0.73 | 5.46 | 0.006602793 |
| Q5JTY5 | CBWD3 | 10.04 | 1.84 | 5.46 | 0.001485959 |
| Q86YP4 | P66A | 9.41 | 1.72 | 5.46 | 1.11267E−05 |
| P29728 | OAS2 | 11.45 | 2.10 | 5.45 | 0.005467741 |
| Q96HL8 | SH3Y1 | 9.37 | 1.72 | 5.45 | 0.008831745 |
| P37198 | NUP62 | 5.67 | 1.04 | 5.44 | 0.009035125 |
| P06493 | CDK1 | 102.61 | 18.91 | 5.43 | 3.72051E−06 |
| Q8N5I2 | ARRD1 | 24.77 | 4.59 | 5.40 | 9.08679E−05 |
| Q99715 | COCA1 | 325.61 | 60.32 | 5.40 | 6.55982E−05 |
| O75381 | PEX14 | 7.50 | 1.39 | 5.38 | 0.005400779 |
| Q5T5P2 | SKT | 3.87 | 0.72 | 5.36 | 0.002176903 |
| Q9BZG1 | RAB34 | 1.46 | 0.27 | 5.35 | 0.009104776 |
| Q9H6R4 | NOL6 | 14.77 | 2.76 | 5.35 | 3.97778E−05 |
| P08637 | FCG3A | 22.89 | 4.29 | 5.34 | 0.000742511 |
| P32322 | P5CR1 | 149.95 | 28.13 | 5.33 | 3.58843E−06 |
| Q9H2H9 | S38A1 | 11.67 | 2.21 | 5.29 | 0.006998838 |
| Q9H3R2 | MUC13 | 64.75 | 12.27 | 5.28 | 0.00415667 |
| Q9H1K1 | ISCU | 1.98 | 0.38 | 5.27 | 0.005766541 |
| Q5JTH9 | RRP12 | 9.61 | 1.83 | 5.26 | 0.00011316 |
| Q9NXW2 | DJB12 | 11.53 | 2.19 | 5.26 | 0.001402788 |
| Q6Y7W6 | PERQ2 | 32.73 | 6.25 | 5.24 | 1.87603E−06 |
| O95801 | TTC4 | 14.28 | 2.73 | 5.23 | 0.004147939 |
| Q8WUM0 | NU133 | 18.90 | 3.61 | 5.23 | 0.000622929 |
| Q9Y508 | RN114 | 61.91 | 11.84 | 5.23 | 0.000319052 |
| Q92797 | SYMPK | 13.17 | 2.53 | 5.21 | 0.003971241 |
| Q8IWA0 | WDR75 | 26.70 | 5.15 | 5.19 | 0.000586437 |
| O60879 | DIAP2 | 10.90 | 2.10 | 5.18 | 0.003681397 |
| P37268 | FDFT | 46.66 | 9.00 | 5.18 | 0.000227797 |
| Q96GW9 | SYMM | 8.24 | 1.59 | 5.18 | 0.000139173 |
| Q9P210 | CPSF2 | 10.06 | 1.95 | 5.17 | 1.80412E−05 |
| P31641 | SC6A6 | 4.90 | 0.95 | 5.17 | 0.003468938 |
| P06400 | RB | 5.87 | 1.14 | 5.16 | 0.005598094 |
| P05981 | HEPS | 17.25 | 3.35 | 5.16 | 7.1056E−05 |
| Q9BWJ5 | SF3B5 | 114.66 | 22.28 | 5.15 | 0.000906386 |
| Q9H5Q4 | TFB2M | 11.51 | 2.25 | 5.13 | 0.00389446 |
| Q8TDD1 | DDX54 | 36.44 | 7.13 | 5.11 | 3.09366E−10 |
| Q9BZX2 | UCK2 | 9.79 | 1.92 | 5.11 | 0.001477202 |
| Q969S3 | ZN622 | 6.09 | 1.19 | 5.10 | 0.006517608 |
| Q9Y2C3 | B3GT5 | 41.14 | 8.08 | 5.09 | 4.34699E−07 |
| O60941 | DTNB | 12.49 | 2.45 | 5.09 | 0.003958744 |
| Q00653 | NFKB2 | 10.37 | 2.05 | 5.07 | 7.67645E−06 |
| Q6U841 | S4A10 | 12.80 | 2.53 | 5.07 | 0.000164601 |
| O95347 | SMC2 | 4.82 | 0.95 | 5.06 | 0.000852265 |
| Q9NYH9 | UTP6 | 9.27 | 1.84 | 5.05 | 0.002314162 |
| O75691 | UTP20 | 7.13 | 1.42 | 5.03 | 0.000530795 |
| Q96C36 | P5CR2 | 77.06 | 15.31 | 5.03 | 1.18346E−05 |
| Q14CX7 | NAA25 | 2.22 | 0.44 | 5.03 | 0.004549945 |
| Q9BQ13 | KCD14 | 11.51 | 2.29 | 5.02 | 0.002770734 |
| Q96GX2 | A7L3B | 38.00 | 7.59 | 5.01 | 5.58179E−05 |
| Q5SRE7 | PHYD1 | 2.18 | 0.44 | 5.00 | 0.003571748 |
| Q9UHI6 | DDX20 | 3.36 | 0.67 | 5.00 | 0.00072136 |
| Q8IYD1 | ERF3B | 45.21 | 9.05 | 4.99 | 2.90165E−07 |
| Q07075 | AMPE | 21.35 | 4.28 | 4.99 | 0.002388481 |
| Q13206 | DDX10 | 63.79 | 12.80 | 4.98 | 2.66412E−05 |
| Q9H0D6 | XRN2 | 52.12 | 10.48 | 4.97 | 4.07852E−07 |
| Q14839 | CHD4 | 60.32 | 12.14 | 4.97 | 5.89237E−05 |
| Q99459 | CDC5L | 19.46 | 3.92 | 4.97 | 0.002636974 |
| P0CB38 | PAB4L | 8.39 | 1.69 | 4.96 | 0.000138513 |
| Q9H307 | PININ | 36.49 | 7.37 | 4.95 | 1.04758E−05 |
| Q8NHP6 | MSPD2 | 1.73 | 0.35 | 4.95 | 0.002215223 |
| Q9BX10 | GTPB2 | 10.35 | 2.10 | 4.92 | 0.000359322 |
| Q9Y2P8 | RCL1 | 23.16 | 4.71 | 4.92 | 1.48301E−06 |
| P33992 | MCM5 | 77.65 | 15.83 | 4.91 | 8.27388E−06 |
| O76031 | CLPX | 17.32 | 3.53 | 4.90 | 0.00038104 |
| Q9Y2X7 | GIT1 | 5.48 | 1.12 | 4.88 | 0.000544104 |
| Q96DH6 | MSI2H | 16.59 | 3.41 | 4.86 | 0.010245618 |
| Q5QJE6 | TDIF2 | 2.29 | 0.47 | 4.86 | 0.004589457 |
| P28340 | DPOD1 | 8.47 | 1.75 | 4.85 | 0.000686267 |
| Q13610 | PWP1 | 12.81 | 2.65 | 4.83 | 0.002086329 |
| Q7L211 | ABHDD | 24.74 | 5.12 | 4.83 | 0.000123587 |
| Q63ZY3 | KANK2 | 10.61 | 2.20 | 4.83 | 0.010189877 |
| Q15042 | RB3GP | 10.65 | 2.22 | 4.81 | 0.000108862 |
| Q8WTT2 | NOC3L | 6.27 | 1.30 | 4.81 | 0.001935223 |
| Q9NVM6 | DJC17 | 9.33 | 1.95 | 4.80 | 0.003754611 |
| P56182 | RRP1 | 111.81 | 23.39 | 4.78 | 1.11057E−06 |
| O15042 | SR140 | 39.72 | 8.31 | 4.78 | 1.34552E−06 |
| O14497 | ARI1A | 3.03 | 0.64 | 4.75 | 0.000407875 |
| P25205 | MCM3 | 104.91 | 22.14 | 4.74 | 2.01582E−05 |
| Q96D46 | NMD3 | 5.66 | 1.20 | 4.73 | 0.002021966 |
| Q8WUA4 | TF3C2 | 7.49 | 1.59 | 4.72 | 0.000695608 |
| Q9Y5Y5 | PEX16 | 10.77 | 2.29 | 4.70 | 0.002137976 |
| P39748 | FEN1 | 62.99 | 13.42 | 4.69 | 5.43605E−06 |
| Q9NR30 | DDX21 | 14.75 | 3.14 | 4.69 | 0.003534444 |
| Q6P1L8 | RM14 | 49.59 | 10.62 | 4.67 | 0.00045561 |
| Q12788 | TBL3 | 51.40 | 11.02 | 4.66 | 6.43077E−06 |
| Q14669 | TRIPC | 7.71 | 1.65 | 4.66 | 0.000817112 |
| P52594 | AGFG1 | 4.53 | 0.98 | 4.65 | 0.010401023 |
| Q8N392 | RHG18 | 19.00 | 4.09 | 4.64 | 0.000187169 |
| P26358 | DNMT1 | 15.21 | 3.28 | 4.64 | 0.00063548 |
| Q9NQA3 | WASH6 | 7.29 | 1.57 | 4.64 | 0.002178484 |
| O75822 | EIF3J | 50.37 | 10.88 | 4.63 | 3.47401E−05 |
| Q0VDF9 | HSP7E | 5.31 | 1.15 | 4.62 | 0.008381011 |
| Q9BY42 | RTF2 | 10.29 | 2.23 | 4.61 | 0.006982497 |
| Q92990 | GLMN | 16.05 | 3.49 | 4.60 | 1.65475E−05 |
| P22676 | CALB2 | 1.76 | 0.38 | 4.57 | 0.00978723 |
| Q9UK59 | DBR1 | 12.20 | 2.67 | 4.57 | 2.05458E−05 |
| Q15269 | PWP2 | 27.51 | 6.04 | 4.55 | 0.000285023 |
| P33527 | MRP1 | 30.96 | 6.83 | 4.53 | 0.000780725 |
| Q9BWU0 | NADAP | 16.51 | 3.65 | 4.53 | 0.002324184 |
| O00425 | IF2B3 | 13.77 | 3.04 | 4.53 | 0.002192508 |
| P49736 | MCM2 | 80.51 | 17.83 | 4.51 | 1.74726E−05 |
| Q9NUQ3 | TXLNG | 43.33 | 9.61 | 4.51 | 0.006878245 |
| P09234 | RU1C | 21.71 | 4.82 | 4.51 | 0.00639531 |
| Q9NUP1 | BL1S4 | 13.86 | 3.08 | 4.50 | 0.000118909 |
| Q16880 | CGT | 6.81 | 1.51 | 4.50 | 0.006588344 |
| Q8IY37 | DHX37 | 78.24 | 17.42 | 4.49 | 0.000416519 |
| P16949 | STMN1 | 133.79 | 29.81 | 4.49 | 6.22591E−08 |
| Q6ZMZ3 | SYNE3 | 4.12 | 0.92 | 4.49 | 0.001218866 |
| Q12800 | TFCP2 | 3.81 | 0.85 | 4.49 | 0.004385402 |
| P50281 | MMP14 | 12.41 | 2.77 | 4.48 | 0.000143231 |
| Q9NRG9 | AAAS | 16.98 | 3.79 | 4.48 | 3.55714E−05 |
| Q9C0J8 | WDR33 | 3.55 | 0.79 | 4.46 | 0.009945866 |
| Q9UL03 | INT6 | 11.46 | 2.58 | 4.44 | 0.000572507 |
| Q5JRA6 | MIA3 | 18.43 | 4.15 | 4.44 | 0.005684898 |
| Q9NY93 | DDX56 | 10.62 | 2.40 | 4.43 | 0.000385047 |
| Q9NZT2 | OGFR | 10.33 | 2.33 | 4.43 | 0.001547502 |
| Q01118 | SCN7A | 13.63 | 3.08 | 4.42 | 0.001076507 |
| Q8NC51 | PAIRB | 37.49 | 8.48 | 4.42 | 1.70054E−05 |
| Q99543 | DNJC2 | 14.03 | 3.18 | 4.42 | 9.5474E−07 |
| Q8WXX5 | DNJC9 | 6.14 | 1.40 | 4.39 | 5.46899E−05 |
| Q6PKG0 | LARP1 | 10.31 | 2.35 | 4.39 | 0.009271221 |
| O94923 | GLCE | 45.11 | 10.29 | 4.38 | 0.00328052 |
| Q8ND04 | SMG8 | 1.80 | 0.41 | 4.38 | 0.00154159 |
| O75936 | BODG | 11.98 | 2.74 | 4.37 | 0.001143365 |
| Q7L592 | NDUF7 | 44.65 | 10.24 | 4.36 | 0.002046407 |
| Q8WVM7 | STAG1 | 4.54 | 1.04 | 4.35 | 0.006637518 |
| Q92598 | HS105 | 287.83 | 66.27 | 4.34 | 7.41055E−08 |
| O14647 | CHD2 | 21.04 | 4.85 | 4.34 | 0.00054177 |
| Q8N9N2 | ASCC1 | 164.61 | 37.94 | 4.34 | 2.75279E−06 |
| Q7Z7F7 | RM55 | 42.65 | 9.85 | 4.33 | 0.003366827 |
| Q7L5A8 | FA2H | 10.72 | 2.48 | 4.32 | 0.001293028 |
| Q9UBB5 | MBD2 | 6.43 | 1.49 | 4.31 | 0.004193671 |
| Q96SB4 | SRPK1 | 7.09 | 1.65 | 4.30 | 0.008498092 |
| Q9BTE7 | DCNL5 | 2.37 | 0.55 | 4.29 | 0.004447004 |
| O00541 | PESC | 47.71 | 11.16 | 4.27 | 8.68858E−06 |
| P19525 | E2AK2 | 41.86 | 9.82 | 4.26 | 0.000111245 |
| P49913 | CAMP | 95.30 | 22.39 | 4.26 | 0.001722057 |
| Q14C86 | GAPD1 | 13.13 | 3.09 | 4.25 | 0.00313657 |
| Q9H0S4 | DDX47 | 8.71 | 2.05 | 4.24 | 0.007558439 |
| Q9UKF6 | CPSF3 | 15.60 | 3.68 | 4.24 | 1.30499E−05 |
| Q9BYG3 | MK67I | 36.86 | 8.70 | 4.24 | 1.60594E−05 |
| P05067 | A4 | 11.04 | 2.61 | 4.24 | 0.001450788 |
| P60201 | MYPR | 29.15 | 6.89 | 4.23 | 0.000682134 |
| Q9NWT1 | PK1IP | 6.91 | 1.63 | 4.23 | 0.009330853 |
| Q9H7B2 | RPF2 | 14.91 | 3.53 | 4.22 | 0.007006263 |
| P41218 | MNDA | 112.34 | 26.71 | 4.21 | 0.00018813 |
| Q12873 | CHD3 | 11.32 | 2.70 | 4.20 | 0.000244642 |
| Q9HA77 | SYCM | 32.19 | 7.69 | 4.19 | 0.000662243 |
| P45973 | CBX5 | 38.39 | 9.19 | 4.18 | 0.000569284 |
| Q15386 | UBE3C | 4.86 | 1.17 | 4.16 | 0.010015749 |
| P01033 | TIMP1 | 35.00 | 8.41 | 4.16 | 9.95103E−05 |
| Q04724 | TLE1 | 8.25 | 1.98 | 4.16 | 0.002682056 |
| Q96T23 | RSF1 | 16.76 | 4.03 | 4.15 | 0.000469251 |
| P13995 | MTDC | 11.41 | 2.75 | 4.15 | 0.005020861 |
| Q9C0C2 | TB182 | 24.66 | 5.95 | 4.14 | 0.001764738 |
| P18583 | SON | 2.39 | 0.58 | 4.13 | 0.004906063 |
| Q9UPE1 | SRPK3 | 5.53 | 1.34 | 4.13 | 0.000253571 |
| Q05DH4 | F16A1 | 37.90 | 9.18 | 4.13 | 1.07771E−05 |
| P49459 | UBE2A | 10.35 | 2.51 | 4.12 | 0.006880563 |
| Q9UBU9 | NXF1 | 10.55 | 2.57 | 4.10 | 0.000844574 |
| Q9UJX2 | CDC23 | 9.91 | 2.43 | 4.07 | 0.008031493 |
| Q9Y5S8 | NOX1 | 24.95 | 6.13 | 4.07 | 0.001916304 |
| Q96TA2 | YMEL1 | 17.31 | 4.26 | 4.07 | 0.000413817 |
| C4AMC7 | WASH3 | 2.23 | 0.55 | 4.06 | 0.005922276 |
| P55196 | AFAD | 22.72 | 5.59 | 4.06 | 0.001625686 |
| Q9UHF1 | EGFL7 | 36.43 | 9.00 | 4.05 | 3.75581E−06 |
| Q5T8P6 | RBM26 | 6.74 | 1.68 | 4.02 | 0.00044417 |
| Q9BUB7 | TMM70 | 36.84 | 9.19 | 4.01 | 0.000542314 |
| Q9UHK6 | AMACR | 48.89 | 12.20 | 4.01 | 0.006726016 |
| P00488 | F13A | 81.47 | 328.38 | 0.25 | 3.03308E−06 |
| P07585 | PGS2 | 397.65 | 1621.39 | 0.25 | 3.96013E−07 |
| Q9Y4J8 | DTNA | 11.59 | 48.51 | 0.24 | 0.00432281 |
| P23141 | EST1 | 42.56 | 178.75 | 0.24 | 8.38461E−05 |
| P54289 | CA2D1 | 7.53 | 31.96 | 0.24 | 0.002135 |
| Q16853 | AOC3 | 224.88 | 961.63 | 0.23 | 2.66363E−11 |
| O43294 | TGFI1 | 31.63 | 135.42 | 0.23 | 0.002452735 |
| Q9NZN4 | EHD2 | 192.60 | 830.19 | 0.23 | 1.36477E−06 |
| P50895 | BCAM | 8.91 | 38.77 | 0.23 | 1.42861E−06 |
| P07327 | ADH1A | 299.62 | 1311.34 | 0.23 | 1.10213E−11 |
| P51178 | PLCD1 | 3.47 | 15.50 | 0.22 | 0.001287748 |
| P09038 | FGF2 | 3.48 | 15.57 | 0.22 | 0.000124018 |
| Q6YHK3 | CD109 | 1.90 | 8.56 | 0.22 | 0.006240587 |
| Q15645 | PCH2 | 7.39 | 34.00 | 0.22 | 0.004054426 |
| P25189 | MYP0 | 3.06 | 14.14 | 0.22 | 0.00595874 |
| P17661 | DESM | 889.44 | 4104.88 | 0.22 | 0.001192879 |
| O75106 | AOC2 | 25.65 | 120.98 | 0.21 | 4.57783E−09 |
| Q92629 | SGCD | 15.21 | 73.25 | 0.21 | 2.07922E−05 |
| Q6UWM9 | UD2A3 | 13.32 | 64.33 | 0.21 | 0.003322962 |
| Q9NZM3 | ITSN2 | 2.42 | 11.88 | 0.20 | 0.005394712 |
| Q02952 | AKA12 | 7.04 | 34.82 | 0.20 | 0.003337753 |
| P08185 | CBG | 2.48 | 12.35 | 0.20 | 0.000215349 |
| Q96P11 | NSUN5 | 0.83 | 4.14 | 0.20 | 0.00738041 |
| Q99969 | RARR2 | 3.15 | 16.03 | 0.20 | 0.004768426 |
| P41235 | HNF4A | 9.71 | 49.63 | 0.20 | 0.005643643 |
| Q9NP58 | ABCB6 | 10.86 | 56.27 | 0.19 | 0.000587634 |
| Q9NUT2 | ABCB8 | 7.98 | 41.65 | 0.19 | 0.002897897 |
| P29536 | LMOD1 | 35.46 | 186.10 | 0.19 | 0.000365793 |
| Q12929 | EPS8 | 2.64 | 14.03 | 0.19 | 0.009238808 |
| Q5T7N7 | F27E1 | 91.35 | 488.42 | 0.19 | 0.004345363 |
| P00325 | ADH1B | 333.11 | 1786.96 | 0.19 | 3.28323E−12 |
| O00533 | NCHL1 | 2.00 | 10.77 | 0.19 | 0.000356478 |
| Q9NRW4 | DUS22 | 1.05 | 5.81 | 0.18 | 0.000335951 |
| Q8WVB6 | CTF18 | 20.12 | 112.56 | 0.18 | 0.000241624 |
| Q13642 | FHL1 | 110.95 | 624.31 | 0.18 | 9.8022E−08 |
| Q5ZPR3 | CD276 | 5.52 | 31.35 | 0.18 | 0.007740115 |
| O00339 | MATN2 | 2.23 | 12.77 | 0.18 | 0.000211092 |
| P55268 | LAMB2 | 26.54 | 151.85 | 0.17 | 0.000112681 |
| Q15648 | MED1 | 1.34 | 7.74 | 0.17 | 0.004779449 |
| Q15746 | MYLK | 43.96 | 254.44 | 0.17 | 0.002030219 |
| P00746 | CFAD | 12.30 | 71.43 | 0.17 | 8.41369E−07 |
| P15088 | CBPA3 | 64.79 | 389.31 | 0.17 | 3.60289E−08 |
| P50440 | GATM | 1.76 | 10.62 | 0.17 | 0.000689397 |
| Q9BX66 | SRBS1 | 43.80 | 273.54 | 0.16 | 0.002178058 |
| Q9BST9 | RTKN | 9.15 | 57.82 | 0.16 | 0.00738038 |
| P08493 | MGP | 10.10 | 64.40 | 0.16 | 0.000181951 |
| P20774 | MIME | 116.54 | 750.25 | 0.16 | 0.000232827 |
| P35625 | TIMP3 | 5.20 | 34.19 | 0.15 | 0.00239417 |
| Q9HBL0 | TENS1 | 26.84 | 184.86 | 0.15 | 0.004431653 |
| Q9BZZ2 | SN | 1.37 | 9.55 | 0.14 | 0.003609636 |
| O43745 | CHP2 | 2.36 | 17.08 | 0.14 | 0.00200161 |
| P02511 | CRYAB | 71.24 | 536.61 | 0.13 | 1.09093E−07 |
| O43556 | SGCE | 1.39 | 10.49 | 0.13 | 0.00117546 |
| P23946 | CMA1 | 90.84 | 694.60 | 0.13 | 6.41727E−09 |
| Q7Z5L7 | PODN | 1.70 | 13.28 | 0.13 | 0.002979922 |
| P51911 | CNN1 | 226.11 | 1786.62 | 0.13 | 0.0058422 |
| Q8NFI3 | ENASE | 0.50 | 4.02 | 0.13 | 0.000425479 |
| Q6ZMJ2 | SCAR5 | 0.31 | 2.52 | 0.12 | 0.007244391 |
| Q14157 | UBP2L | 10.11 | 82.19 | 0.12 | 0.005240751 |
| P22748 | CAH4 | 4.96 | 41.20 | 0.12 | 0.008863677 |
| Q9Y6R1 | S4A4 | 6.10 | 50.75 | 0.12 | 3.696E−05 |
| P30533 | AMRP | 0.70 | 5.90 | 0.12 | 8.95247E−06 |
| P47989 | XDH | 0.62 | 5.53 | 0.11 | 0.000933962 |
| Q13361 | MFAP5 | 18.69 | 166.61 | 0.11 | 3.60808E−06 |
| Q16647 | PTGIS | 5.71 | 51.20 | 0.11 | 0.001005471 |
| P46821 | MAP1B | 1.12 | 10.05 | 0.11 | 0.00417868 |
| Q9BXN1 | ASPN | 40.93 | 376.88 | 0.11 | 5.45636E−06 |
| P51888 | PRELP | 96.58 | 909.16 | 0.11 | 2.72175E−08 |
| O14578 | CTRO | 0.09 | 0.85 | 0.10 | 0.009783761 |
| O15394 | NCAM2 | 12.51 | 123.83 | 0.10 | 1.68628E−07 |
| P30825 | CTR1 | 3.22 | 32.19 | 0.10 | 8.66602E−09 |
| Q14978 | NOLC1 | 65.58 | 664.78 | 0.10 | 3.67207E−05 |
| Q99797 | MIPEP | 0.22 | 2.25 | 0.10 | 0.001570675 |
| Q14714 | SSPN | 1.33 | 15.04 | 0.09 | 0.000206126 |
| P22105 | TENX | 5.28 | 59.86 | 0.09 | 1.92842E−08 |
| P13591 | NCAM1 | 4.55 | 51.68 | 0.09 | 3.67209E−08 |
| P14207 | FOLR2 | 1.22 | 13.85 | 0.09 | 0.003011148 |
| Q8IXT5 | RB12B | 6.97 | 84.19 | 0.08 | 0.000110105 |
| Q6UXI9 | NPNT | 0.36 | 4.41 | 0.08 | 0.004299846 |
| Q9NY27 | PP4R2 | 1.00 | 13.03 | 0.08 | 8.49984E−06 |
| Q13501 | SQSTM | 1.81 | 26.14 | 0.07 | 0.000683456 |
| Q9BZQ8 | NIBAN | 3.97 | 58.89 | 0.07 | 0.001725005 |
| O76038 | SEGN | 1.37 | 20.50 | 0.07 | 3.43246E−05 |
| Q5VV42 | CDKAL | 0.38 | 5.90 | 0.07 | 0.00394269 |
| Q96AY3 | FKB10 | 4.08 | 63.25 | 0.06 | 4.82232E−06 |
| P11532 | DMD | 1.30 | 20.45 | 0.06 | 0.000188968 |
| Q6VN20 | RBP10 | 5.91 | 94.49 | 0.06 | 0.000445205 |
| Q04726 | TLE3 | 0.39 | 6.57 | 0.06 | 0.001518459 |
| Q641Q2 | FA21A | 5.26 | 96.82 | 0.05 | 9.98106E−05 |
| A6NKC4 | FCGRC | 3.18 | 58.78 | 0.05 | 0.007997391 |
| Q9Y496 | KIF3A | 0.95 | 17.99 | 0.05 | 9.27913E−05 |
| Q2UY09 | COSA1 | 0.71 | 13.79 | 0.05 | 0.000490481 |
| Q8N468 | MFSD4 | 0.15 | 2.94 | 0.05 | 0.00630615 |
| P05162 | LEG2 | 1.55 | 32.35 | 0.05 | 0.006289165 |
| P45381 | ACY2 | 0.73 | 15.52 | 0.05 | 3.16166E−05 |
| Q9NXH9 | TRM1 | 3.70 | 92.51 | 0.04 | 6.46068E−09 |
| Q13683 | ITA7 | 0.79 | 19.92 | 0.04 | 0.001277399 |
| Q8WW12 | PCNP | 0.07 | 1.79 | 0.04 | 0.001375166 |
| P10915 | HPLN1 | 0.88 | 23.02 | 0.04 | 0.000304764 |
| Q96P44 | COLA1 | 0.16 | 4.45 | 0.04 | 0.009688581 |
| Q9H4A3 | WNK1 | 1.83 | 50.88 | 0.04 | 1.55114E−05 |
| P08319 | ADH4 | 0.63 | 18.03 | 0.03 | 0.000857797 |
| Q92633 | LPAR1 | 1.00 | 29.82 | 0.03 | 1.20187E−05 |
| O94911 | ABCA8 | 1.00 | 29.94 | 0.03 | 9.50584E−06 |
| Q15493 | RGN | 0.25 | 7.53 | 0.03 | 0.002720973 |
| P11388 | TOP2A | 10.41 | 327.97 | 0.03 | 4.4869E−06 |
| P06276 | CHLE | 0.34 | 11.68 | 0.03 | 5.43531E−06 |
| Q9BWE0 | REPI1 | 0.62 | 21.63 | 0.03 | 0.001012654 |
| Q8NB16 | MLKL | 0.63 | 22.94 | 0.03 | 1.22645E−05 |
| Q9UFC0 | LRWD1 | 0.61 | 22.51 | 0.03 | 0.000359656 |
| Q7Z7G0 | TARSH | 0.77 | 28.53 | 0.03 | 3.65857E−06 |
| Q8NCG7 | DGLB | 0.11 | 7.22 | 0.02 | 2.46766E−07 |
| Q96GM8 | TOE1 | 0.39 | 26.27 | 0.01 | 6.93514E−05 |
| Q6WCQ1 | MPRIP | 0.20 | 15.80 | 0.01 | 0.0017953 |
| P78539 | SRPX | 0.00 | 9.50 | 0.00 | 0.004543364 |
| P27930 | IL1R2 | 0.00 | 1.55 | 0.00 | 0.009975281 |
| Q13491 | GPM6B | 0.00 | 9.01 | 0.00 | 0.002413876 |
| Q12860 | CNTN1 | 0.00 | 3.57 | 0.00 | 6.54438E−05 |
| O43895 | XPP2 | 0.00 | 2.34 | 0.00 | 0.003042904 |
| Q9BX67 | JAM3 | 0.00 | 4.03 | 0.00 | 0.003016704 |
| O00501 | CLD5 | 0.00 | 4.99 | 0.00 | 0.006191187 |
| P32004 | L1CAM | 0.00 | 7.95 | 0.00 | 1.07487E−05 |
| Q7Z3B1 | NEGR1 | 0.00 | 7.84 | 0.00 | 0.000809911 |
| Q14160 | SCRIB | 0.00 | 1.41 | 0.00 | 0.005971086 |
| Q8TB72 | PUM2 | 0.00 | 4.33 | 0.00 | 0.001778617 |
| Q8NFZ8 | CADM4 | 0.00 | 3.98 | 0.00 | 0.004491047 |
| Q9BTC0 | DIDO1 | 0.00 | 23.90 | | 0.006671581 |

TABLE 3
A list of description for a panel of biomarkers

| UniProt | Accession | Gene | Protein |
|---|---|---|---|
| ID | No. | Symbols | Description |
| P23946 | 126825 | CAM1 | Chymase |
| P15088 | 317373331 | CPA3 | Mast cell |
| | | | carboxypeptidase A |
| Q6UX06 | 74749412 | OLM4 | Olfactomedin-4 |
| O00515 | 206729878 | LAD1 | Ladinin-1 |
| P16444 | 92090943 | DPEP1 | Dipeptidase 1 |
| Q9NZT2 | 146331047 | OGFR | Opioid growth |
| | | | factor receptor |
| P54753 | 76803655 | EPHB3 | Ephrin type-B |
| | | | receptor 3 |
| Q9Y446 | 20139301 | PKP3 | Plakophilin-3 |
| P40199 | 296439410 | CEAM6 | Carcinoembryonic |
| | | | antigen-related cell |
| | | | adhesion molecule 6 |
| P36952 | 229462757 | SERPINB5 | Serpin B5 |
| Q9H3R2 | 635377434 | MUC13 | Mucin-13 |

Here we have showed that a comprehensive CRC proteome map can be characterized by analyses of paired tumor and adjacent normal tissue samples using a standardized proteomics workflow and a novel pathway analysis strategy. Our data demonstrated that the abundance alteration in a group of proteins (responsible for a specific cellular function or process) instead of only individual proteins could be the major contributor of CRC, and provided evidence to interpret how a dozen or a few dozen mutated tumor driver genes facilitate uncontrolled cancer cell growth and invasion. In CRC, the mutations in APC, p53, and k-Ras, or chromosomal instability and microsatellite instability events may initiate changes of gene expression. As a result, these changes lead to significant elevations of proteins required for assembling chromatin modification, DNA replication and damage repair, and transcription and translation machinery, which in-turn fuel the proliferation of tumor cells eventually.

Essentially our findings suggest a proteomic “teeterboard” mechanism for the regulation of pathways by modulating the balance between inhibitory regulators and activating regulators. In cells, the activations of signaling pathways orchestrate the regulation of cell fate, cell survival, apoptosis, and cell proliferation; the regulation of signaling pathways and the transduction of signals are integrated in the pathway components, which could be functionally divided into inhibitory regulators and activating regulators. The balance of the two major components determines the pathway activation status. During tumorigenesis the decreased expression of inhibitory regulators and increased expression of activating regulators break off the well-organized/programmed cellular regulation network. Therefore, we propose a tumorigenesis model: molecular malfunction events including tumor driver genes' mutations, chromosomal instability and microsatellite instability initiate changes of gene expression, which lead to decreased expression of inhibitory molecules but increased expression of activating regulators to reactivate silenced pathways, and elevated expression of machinery for chromatin modification, DNA replication and damage repair, transcription and translation, altogether affording a proliferative advantage. This process was accurately reflected by the proteomic abnormality observed in cancer tissues in this study.

A panel of 11 proteins, which includes Chymase (CAM1), Mast cell carboxypeptidase A (CPA3), Olfactomedin-4 (OLM4), Ladinin-1 (LDA1), Dipeptidase-1 (DPEP1), Opioid growth factor receptor (OGFR), Ephrin type-B receptor 3 (EPHB3), Plakophilin-3 (PKP3), Carcinoembryonic antigen-related cell adhesion molecule 6 (CEAM6), SerpinB5 (SERPINB5), and Mucin-13 (MUC13), is selected as CRC protein biomarkers to comprehensively distinguish tumor from normal colorectal tissue and determine the tumor lymphatic invasion status. Two enzymes, mast cell carboxypeptidase A and chymase secreted by mast cells are significantly diminished in CRC which is used as two positive markers for normal colorectal tissue. The other nine proteins including CEAM6, SERPINB5, MUC13, OLM4, LAD1, DPEP1, OGFR, EPHB3 and PKP3 are significantly overexpressed in tumor. Based on their relative abundances in tumor cell the 9 protein panel can be used to determine the lymphatic invasion status. The tumor has higher CEAM6, SERPINB5, and MUC13 but relative lower LAD1 and DPEP1 the more it is likely at node-positive disease stage (FIG. 1a).

The instant application discloses a method for determining if a subject has an increased risk having a colorectal disease or disorder comprising:

a) isolating a biological sample containing a test specimen from a biopsy specimen from said subject,

b) isolating a biological sample containing normal colorectal cells or tissue from a biopsy specimen from said subject or a family member of said subject,

c) analyzing protein abundances of biomarkers for the samples from a) and b),

d) comparing the results from c) between the abnormal and normal colorectal cells or tissue.

The instant application discloses a set of reagents to measure the levels of biomarkers in a specimen, wherein the biomarkers are a panel of biomarkers and their measurable fragments: OLM4, LAD1, DPEP1, OGFR, EPHB3, PKP3, CEAM6, SERPINB5 and MUC13 proteins.

### EXAMPLES

Paired CRC and AT specimens were processed for the extraction of total proteins. Equal amounts of protein samples were separated by SDS-PAGE followed by the fractionation of each lane (one sample) into 16 gel slices. The 16 gel slices were further processed for in-gel trypsin digestion to obtain 16 peptide fractions which were analyzed sequentially by LC-MS/MS on a Q-Exactive mass spectrometer equipped with a Dionex Ultimate 3000 RSLCnano system using HCD fragmentation. This resulted in 16 raw MS files from the gel lane of one specimen sample, which were grouped for a database search against the UniProtKB/Swiss-Prot human protein sequence database using SEQUEST and Percolator algorithms in the Thermo Proteome Discoverer 1.4.1 platform to generate a proteome profile. 44 proteome profiles (22 CRC and 22 AT) were generated for 22 paired samples. The relative completeness of the 44 proteome profiles were evaluated using ten groups of well-known “housekeeping” protein complexes consisting of 406 proteins (353 unique proteins and 53 isoforms) as the parameters. A score (0 to 100) was assigned based on the percentage of the 406 “housekeeping” proteins identified. The relative protein abundance in each of 44 proteome profiles was quantified by calculation of the normalized spectral abundance factor (NSAF). In order to quantitatively describe the relative abundance, the ppm (part per million) was chosen as the unit, and the 1,000,000 ppm value was assigned to each proteome profile. A ppm value at the range of 0 to 1,000,000 ppm for each identified protein in each proteome profile was calculated based on its NSAF. The average abundance of each identified protein for CRC and AT was calculated based on 22 CRC proteome profiles and 22 AT proteome profiles, respectively. The comparison between CRC and AT was performed either at a group level using average ppm values and summed values, or at an individual level using individual ppm values.

### Example 1

### Tumor and Adjacent Tissue Samples

All specimens were collected from patients in the Affiliated Hospital of Nantong University (Nantong, China) in accordance with approved human subject guidelines authorized by the Medical Ethics and Human Clinical Trial Committee at the Hospital. Following surgery, the tumor and adjacent normal tissue (AT) specimens were collected in separate tubes, kept in dry ice during transportation, and stored at −80° C. before further processing. AT specimens were obtained from the distal edge of the resection at least 5 cm from the tumor. 22 pairs of cancerous and adjacent normal tissue specimens were collected from 22 individual patients (10 with lymph node metastasis and 12 without lymph node metastasis) (Table 1). All CRC patients had histologically verified adenocarcinoma of the colon or rectum that was confirmed by pathologists. Patient characteristics were obtained from pathology records. Subjects with a history of other malignant diseases or infectious disease, or who had undergone surgery 6 months prior to the start of this research were excluded for this retrospective study.

### Example 2

### Preparation of Protein Extraction, Separation of Proteins, and In-Gel Trypsin Digestion

Total protein extraction from fresh frozen tissue specimens was prepared by the following method. Frozen tissue samples (0.05-0.1 gram) were cut into small pieces (1 mm size) using a clean sharp blade, and transferred into 1.5 ml tubes. A 0.4 ml lysis buffer (20 mM Tris-HCl, pH 7.5, 150 mM NaCl, 1 mM Na₂EDTA, 1 mM EGTA, 1% Triton X-100, Protease inhibitor cocktail pill) was added into each sample tube. The tissues were homogenized using a Dounce homogenizer. After Homogenization, 50 μl of 10% SDS and 50 μl of 1M DTT were added into the mixture followed by incubation at 95° C. for 10 min. After incubation the extraction was sonicated to further breakdown DNA. Sonicated mixtures were centrifuged at 15,000×g for 10 minutes. Supernatants were collected and stored at −80° C. for further analysis. The protein concentration of the supernatants was determined by a BCA™ Reducing Reagent compatible assay kit (Pierce/Thermo Scientific).

Equal amounts of protein (133 μg) from each sample were loaded onto a NuPAGE 4-12% Bis-Tris Gel (Life Technologies). After electrophoresis the gel was stained with SimplyBlue SafeStain (Life Technologies), and subsequently de-stained thoroughly. For preparing in-gel trypsin digested peptides, the de-stained gel was washed with ion-free water three times, and each lane representing one sample was sliced horizontally into 16 slices. Each slice was diced into tiny pieces (1-2 mm) and placed into 1.5 ml centrifuge tubes. Proteins in the gel were treated with DTT for reduction, then iodoacetamide for alkylation, and further digested by trypsin in 25 mM NH₄HCO₃ solution. The digested protein was extracted as described elsewhere. The extracted peptides were dried and reconstituted in 20 μl of 0.1% formic acid before nanospray LC/MS/MS analysis was performed.

### Example 3

### Nanospray LC/MS/MS Analysis

16 tryptic peptide fractions from one specimen sample were analyzed sequentially using a Thermo Scientific Q-Exactive hybrid Quadrupole-Orbitrap Mass Spectrometer equipped with a Thermo Dionex UltiMate 3000 RSLCnano System. Tryptic peptide samples were loaded onto a peptide trap cartridge at a flow rate of 5 μL/min. The trapped peptides were eluted onto a reversed-phase 25 cm C18 PicoFrit column (New Objective, Woburn, Mass.) using a linear gradient of acetonitrile (3-36%) in 0.1% formic acid. The elution duration was 110 min at a flow rate of 0.3 μL/min. Eluted peptides from the PicoFrit column were ionized and sprayed into the mass spectrometer, using a Nanospray Flex Ion Source ES071 (Thermo) under the following settings: spray voltage, 1.6 kV, Capillary temperature, 250° C. The Q Exactive instrument was operated in the data dependent mode to automatically switch between full scan MS and MS/MS acquisition. Survey full scan MS spectra (m/z 300-2000) was acquired in the Orbitrap with 70,000 resolution (m/z 200) after accumulation of ions to a 3×10⁶ target value based on predictive AGC from the previous full scan. Dynamic exclusion was set to 20 s. The 12 most intense multiply-charged ions (z≧2) were sequentially isolated and fragmented in the Axial Higher energy Collision-induced Dissociation (HCD) cell using normalized HCD collision energy at 25% with an AGC target 1e5 and a maxima injection time of 100 ms at 17,500 resolution.

### Example 4

### LC/MS/MS Data Analysis

The raw MS files were analyzed using the Thermo Proteome Discoverer 1.4.1 platform (Thermo Scientific, Bremen, Germany) for peptide identification and protein assembly. For each specimen sample, 16 raw MS files obtained from 16 sequential LC-MS analyses were grouped for a single database search against the Human UniProtKB/Swiss-Prot human protein sequence databases (20597 entries, Dec. 20, 2013) based on the SEQUEST and percolator algorithms through the Proteome Discoverer 1.4.1 platform. Carbamidomethylation of cysteines was set as a fixed modification. The minimum peptide length was specified to be five amino acids. The precursor mass tolerance was set to 15 ppm, whereas fragment mass tolerance was set to 0.05 Da. The maximum false peptide discovery rate was specified as 0.01. The resulting Proteome Discoverer Report contains all assembled proteins (a proteome profile) with peptides sequences and matched spectrum counts. 44 proteome profiles were generated for 22 paired specimen samples (22 CRCs and 22 ATs).

### Example 5

### Protein Quantification

Protein quantification used the normalized spectral abundance factors (NSAFs) method to calculate the protein relative abundance for each identified protein in each proteome profile. In order to quantitatively describe the relative abundance, the ppm (part per million) was chosen as the unit and the 1,000,000 ppm value was assigned to each proteome profile. A ppm value at the range of 0 to 1,000,000 ppm for each identified protein in each proteome profile was calculated based on its normalized NSAF.

The ppm (part per million) was calculated as follow:

RC_{N}=10⁶×NSAF_{N}

### Where:

RC_{N} is the relative concentration of protein N in the proteome of test sample
NSAF_{N} is the protein's normalized spectral abundance Factor
N is the protein index.
Normalized Spectral Abundance Factors (NSAFs) were calculated as follows:

NSAF_{N}=(S_{N}/L_{N})/(Σⁿᵢ₌₁Sᵢ/Lᵢ)

### Where:

N is the protein index
S_{N} is the number of peptide spectra matched to the protein
L_{N} is the length of protein N (number of amino acid residues)
n is the total number of proteins in the input database (proteome profile for one specimen sample).
   We chose to use ppm as the relative unit of protein concentration because the dynamic range for the majority of proteins in a test sample is at least 6 orders of magnitude. Therefore, it is convenient to observe the difference by assigning a value to any identified protein using the ppm. For example,

Histone H4 in AT was 13041±4025 ppm, in CRC was 10903±3821 ppm, GAPDH in AT was 5473±1623 ppm, in CRC was 5932±1480 ppm, and Caspase-8 in AT was 6.1±9.6 ppm, in CRC was 14.6±10.8 ppm. The MEAN, STDEV, T-test values (p-values) were calculated using Microsoft Excel. The ratio of CRC versus AT was defined as 1000 or 0.001 if the protein was not identified in AT or in CRC respectively.

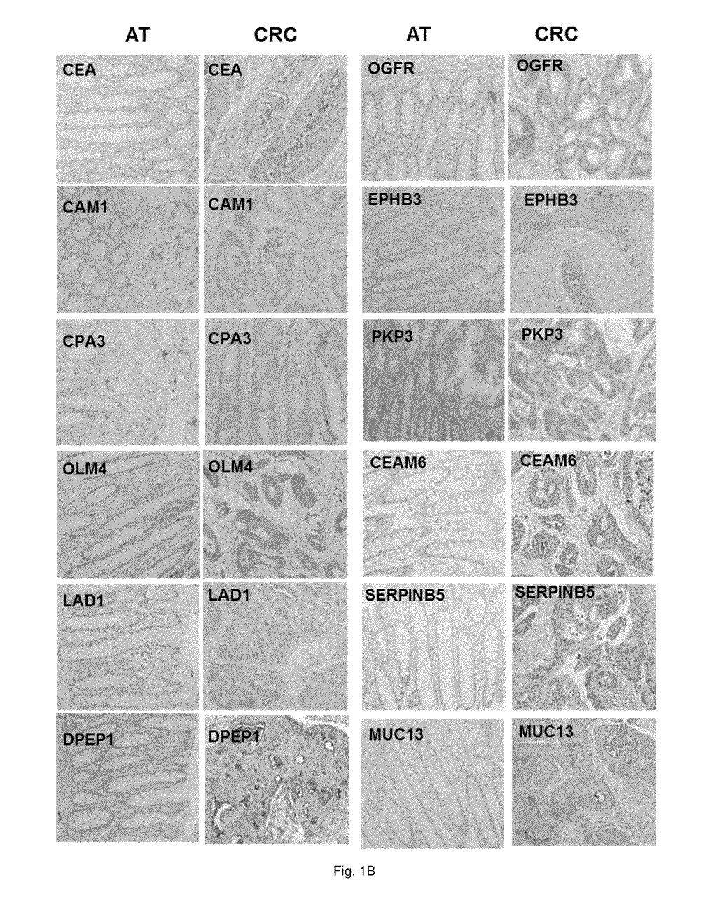

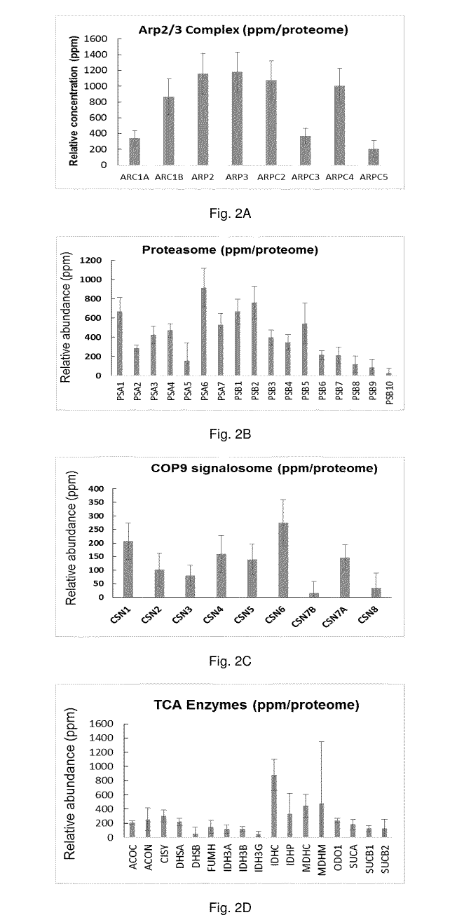

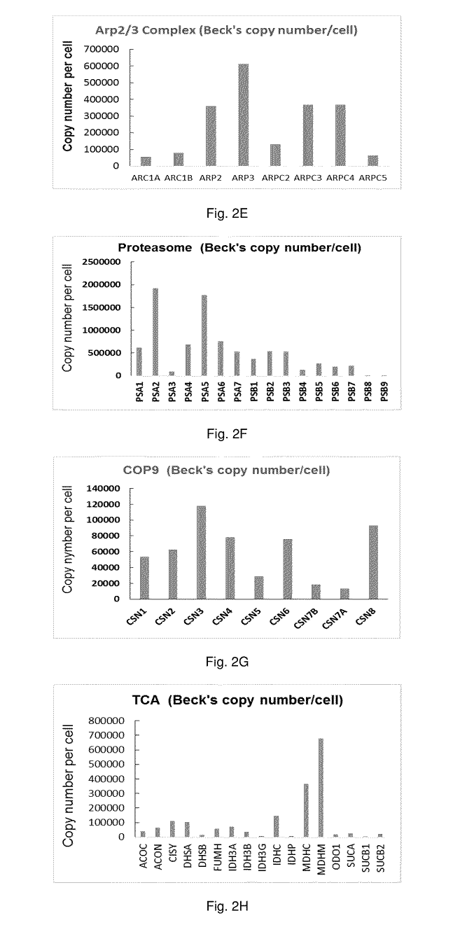

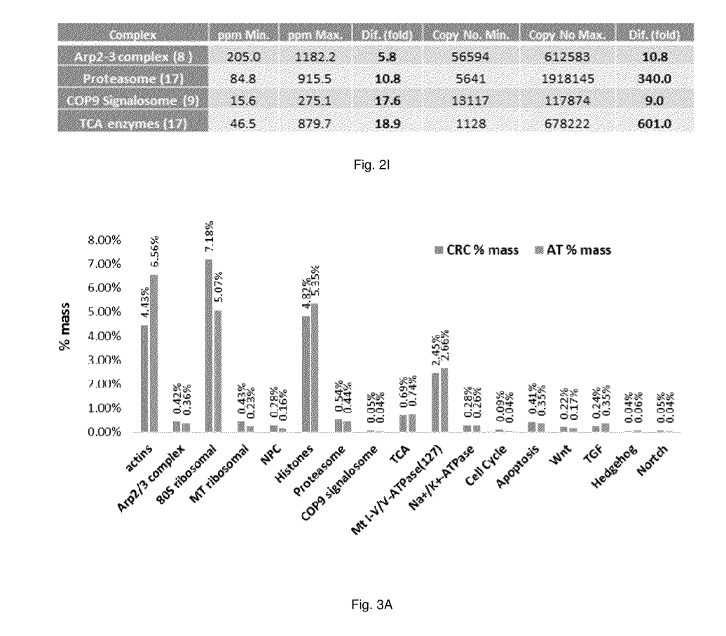

To evaluate the ppm quantification method we compared the relative protein abundance calculated based on NSAFs using ppm as the unit by this study and the published relative abundance calculated in Beck's copy number³⁰. All subunits from four housekeeping protein complexes including the Arp⅔ complex (7 subunits plus one isoform), the COP9 complex (8 subunits plus one isoform), and the Proteasome (17 subunits) and TCA 17 enzymes were used for comparison. As shown in Extended Data FIG. 2 the dynamic range of relative abundance among the members of a complex quantified using ppm was much less than that of Beck's copy number. The dynamic range between the minimum and maximum for tested four complexes was from 5 to 19 fold difference according to spectrum counts based measurements while it was from 9 to 600 fold difference according to published Beck's copy number. This comparison result indicated that the relative protein abundance measured based on spectrum counts quantification would be more accurate or at least a useful alternative.

### Example 6

### Evaluation of the “Quality” of Proteome Profiles

Due to the instrument limitations and wide dynamic range of protein abundances, the most current LC/MS/MS settings are unable to recover the whole proteome, especially the lowest abundance proteins in one experiment. Although it was difficult to obtain a complete proteome from one experiment it was necessary to find an effective approach to evaluate the quality and relative completeness of a set of proteome profiles generated over a period of time before these profiles could be analyzed together unbiasedly. We used the “housekeeping” protein complexes and the distribution of protein population to examine the quality of a proteome profile. It is well-known that “housekeeping” proteins and their complexes are essential for maintaining the life status of a cell, and exist in all tissue/cell types for a life-long time. Therefore, we hypothesized that if these complexes including all subunits could be quantitatively identified and showed no obvious changes between analyses, it indicated that these proteomic profile datasets were relatively complete and comparable, and that the analysis workflow was reliable. Ten groups of well-known “housekeeping” protein complexes consisting of 406 proteins, including 353 unique proteins and 53 isoforms or subtypes (Table 4), were selected as the parameters for the evaluation. The ten groups of complexes were the Arp⅔ complex (8 subunits plus alpha and beta actins), 86 (79 and 7 isoforms) cellular (60S and 40S) ribosomal proteins, 77 mitochondrial (28S and 39S) ribosomal proteins, Nuclear pore complex 38 (34 subunits including GTP-binding nuclear protein Ran, Ran GTPase-activating protein 1 (RAGP1), Ran-specific GTPase-activating protein (RANG), and Ran-binding protein 3 (RANB3), and 4 isoforms), 5 Histones (H1 (5 subtypes), H2A (8 subtypes), H2B (3 subtypes), H3 (4 subtypes) and H4), Proteasome complex (17 subunits), COP9 signalosome complex (9 subunits), TCA enzymes (17 key enzymes), Mitochondrial respiratory chain complexes I-V (94 subunits), and V-type proton (ATPase Complex, 14 subunits consisting 24 isoforms), and Na+/K+-ATPase (sodium-potassium pump, 2 subunits, 7 isoforms). A score (0 to 100) was assigned based on the percentage of the 406 “housekeeping” proteins identified. 44 proteome profiles from this study were scored at an average 92 suggesting these profiles were at the same level of completeness. Three unique proteins, 80S ribosomal protein L41, V-type proton ATPase 21 kDa proteolipid subunit, and V-type proton ATPase subunit e1 or e2, were not identified in this study. To demonstrate the feasibility of this evaluation method we assessed two sets of publically available MS raw data files (http://proteomics.cancer.gov/). One set of 94 MS raw data files (94 CRC samples) from the TCGA-CRC cancer program were scored at an average of 80.3; Another set of 12 MS raw data files from TCGA-Breast cancer program were scored at an average of 98.5.

We next assessed the quality of a proteome profile based on the distribution of its protein population. The distribution of identified proteins per concentration range was analyzed using the Excel-histogram function. The average abundance for each identified protein was calculated as described above. The distribution of all identified 12380 proteins displayed a normal distribution with a major peak and a minor peak representing two populations. The major peak represented 62% (CRC) and 60% (AT) of identified proteins with a relative abundance more than 1 ppm, and the minor peak represented about 38% (CRC) and 40% (AT) of identified proteins with an abundance less than 1 ppm. The majority proteins in the minor peak were randomly identified with one or few PSM across 22 CRC samples or 22 AT samples. To evaluate the method 94 sets of MS raw profiles for 94 CRC samples from the TCGA-CRC cancer program, 12 sets of MS raw datasets from TCGA-Breast cancer program were analyzed. The distributions of identified proteins in 94 TCGA-CRC data files and 12 TCGA-Breast data files were normal distribution and showed the same distribution patterns.

Considering the practical reality, a small panel of protein biomarkers would have more advantages. We identified a panel of 11 proteins based on the relative abundance (mean abundance in CRC >20 ppm) from the ranked 740 proteins to distinguish cancer tissues from normal colorectal tissues obviously (FIG. 1c, Table 2). The expression of CAM1 protein in cancer tissue is decreased by 3-8 fold in comparison with normal tissue. The expression of CPA3 protein in cancer tissue is decreased by 3-6 fold in comparison with normal tissue. The expression of OLM4 protein in cancer tissue is increased by 3-6 fold in comparison with normal tissue. The expression of LAD1 protein in cancer tissue is increased by 10-38 fold in comparison with normal tissue. The expression of DPEP1 protein in cancer tissue is increased by 3-14 fold in comparison with normal tissue. The expression of OGFR protein in cancer tissue is increased by 4-8 fold in comparison with normal tissue. The expression of EPHB3 protein in cancer tissue is increased by 5-11 fold in comparison with normal tissue. The expression of PKP3 protein in cancer tissue is increased by 3-5 fold in comparison with normal tissue. The expression of CEAM6 protein in cancer tissue is increased by 10-28 fold in comparison with normal tissue. The expression of SERPINB5 protein in cancer tissue is increased by 10-25 fold in comparison with normal tissue. The expression of MUC13 protein in cancer tissue is increased by 3-5 fold in comparison with normal tissue.

TABLE 4
A panel of 11 proteins as biomarkers of colorectal cancer

| | CRC | | | | |
|---|---|---|---|---|---|
| Gene | cancer | CRC | Normal | Normal | |
| Symbols | (ppm) | STD | (ppm) | STD | pValue |
| CAM1 | 90.84 | 134.4 | 694.7 | 366.8 | 6.41E−09 |
| CPA3 | 64.78 | 75.22 | 389.3 | 213.5 | 3.60E−08 |
| OLM4 | 433.72 | 457.7 | 74.40 | 83.39 | 0.00078 |
| LAD1 | 67.32 | 77.39 | 1.762 | 5.079 | 0.00028 |
| DPEP1 | 45.90 | 66.33 | 3.247 | 11.54 | 0.0049 |
| OGFR | 39.27 | 29.41 | 4.702 | 6.393 | 3.00E−06 |
| EPHB3 | 21.01 | 28.50 | 1.916 | 2.493 | 0.0032 |
| PKP3 | 111.81 | 70.34 | 23.39 | 19.17 | 1.11E−06 |
| CEAM6 | 79.65 | 83.24 | 2.877 | 4.764 | 9.38E−05 |
| SERPINB5 | 249.3 | 259.1 | 9.854 | 14.13 | 9.11E−05 |
| MUC13 | 64.75 | 80.15 | 12.27 | 13.07 | 0.0042 |

### Example 7

### Pathway Analysis

The cell functions are executed and regulated by the entire sets of proteins (the proteome). The regulation of different cellular functions have been categorized into a number of pathways such as the Wnt signaling pathway and the TGF signaling pathway. In each pathway, the components according to their function are generally named as ligands, receptors, activating regulators, inhibitory regulators, and effectors. In order to measure the activation strength of a pathway, the protein molecules that belong to either ligands, receptors, activating regulators, or inhibitory regulators were grouped and their relative abundances (ppm) were summed. Based on the summed abundance of each grouped components, the activation strength or activation status of a pathway could be compared between two proteome profiles. The proteins list for all analyzed pathways and processes were obtained from the KEGG pathway database and their functional annotation were manually confirmed using the UniProtKB protein database and the NCBI protein database or available publications.

### Example 8

### Immunohistochemistry

Specimens were mounted in paraffin and cut into 8 μm sections. The paraffin sections were treated with xylene and rehydrated. After antigen retrieval, endogenous peroxidase activity was quenched for 30 minutes with 3% H₂O₂ at room temperature. Nonspecific binding sites were blocked by incubation in normal goat serum for 30 minutes at room temperature. Sections were then incubated over-night at 4° C. with primary polyclonal antibodies (10-1000 dilution) including anti-OLFM4, Plakophilin-3, anti-CEAM6, anti-MUC13, anti-CEA, anti-EPH receptor B3, anti-Chymase, anti-CPA3, anti-LAD1, anti-SerpinB5, anti-DPEP1, and anti-OGFR. After the sections were rinsed, a secondary antibody detection Reagent (MaxVisionTM2 kit, Maixin Scientific, China) was incubated at room temperature for 30 minutes. The bound antibody complexes were stained for 5 to 20 minutes with Diaminobenzidine (DAB) and then counterstained with Hematoxylin. Slides were photographed with an Olympus photomicroscope. The results are showed in FIG. 1b.

### Example 9

### Western Blot Analysis

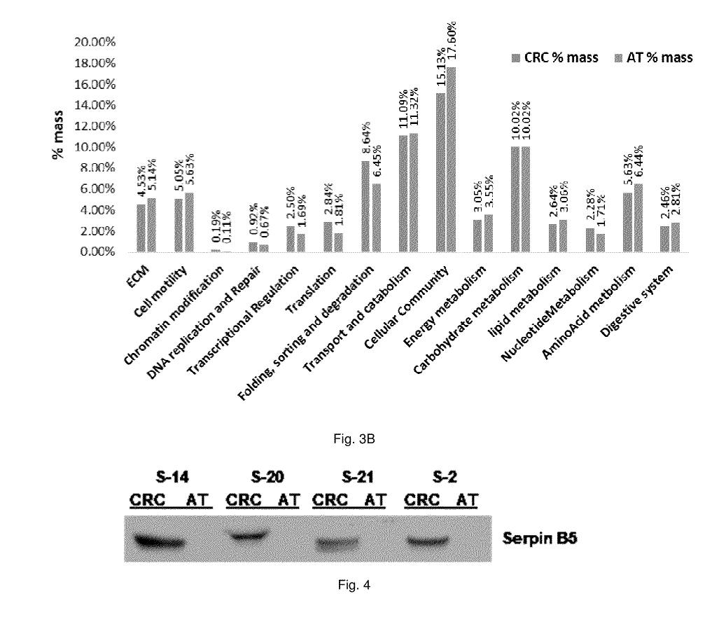

For Western blot analysis equal amount of samples from paired CRC and AT were resolved by 4-12% LDS-NuPAGE gels, transferred to nitrocellulose membranes, and analyzed by western blot (WB) with antibody (100-5000 dilution) selecting from the group consisting of anti-OLFM4, Plakophilin-3, anti-CEAM6, anti-MUC13, anti-CEA, anti-EPH receptor B3, anti-Chymase, anti-CPA3, anti-LAD1, anti-SerpinB5, anti-DPEP1, and anti-OGFR using enhanced chemiluminescence (ECL; Amersham, Piscataway, N.J.). The results are showed in FIG. 4.

### Example 10

### ELISA

Biomarkers' concentrations in plasma/serum specimen were determined using an enzyme-linked immunosorbent assay (ELISA). The samples were analyzed in triplicate and the mean concentrations were calculated. The samples were transferred to 96-well plates coated with primary antibodies (100-5000 dilution) consisting of anti-OLFM4, Plakophilin-3, anti-CEAM6, anti-MUC13, anti-CEA, anti-EPH receptor B3, anti-Chymase, anti-CPA3, anti-LAD1, anti-SerpinB5, anti-DPEP1, and anti-OGFR. Plates were incubated in cold room for 3 hr, after which plates were washed with PBS buffer using an automated plate washer. Luminescence in each well was measured with an Envision plate reader using Gaussia FLEX luciferase kit (New England Biolabs). After luminescence measurement, HRP-conjugated secondary antibody in ELISA buffer (1×PBS, 2% goat serum, 5% Tween 20) was added to wells. Plates were washed in 1×PBS/0.05% Tween 20 with a plate washer and ELISA signal was detected with 3,3′,5,5′-tetramethylbenzidine (TM B) substrate.

### Example 11

### Determination of the Expression Level of 11 Biomarker Panel in Patients by Immunoprecipitation assisted Mass Spectrometry based Quantification

Biomarkers' concentrations in plasma/serum specimen were determined using an immunoprecipitation assisted MS assay. The samples were analyzed in triplicate and the mean concentrations were calculated. The samples were transferred to 1.5 mL tubes with 11 primary antibodies (10-1000 dilution) consisting of anti-OLFM4, Plakophilin-3, anti-CEAM6, anti-MUC13, anti-CEA, anti-EPH receptor B3, anti-Chymase, anti-CPA3, anti-LAD1, anti-SerpinB5, anti-DPEP1, and anti-OGFR which are immobilized on protein G agarose beads/magnetic beads. The reaction mixtures were incubated in cold room for 3 hour to overnight. After incubation the protein G agarose beads conjugated with 11 antibodies were collected by centrifugation and were washed with 1×PBS/0.05% Tween 20. All protein bounded on the protein G agarose beads were quantified by a mass spectrometer.

### Example 12

### Determination of the Expression Level of Biomarker Panel in Different Stages of Colorectal Tumors

Total protein extraction from fresh frozen tissue specimens was prepared by the following method. Frozen tissue samples (0.05-0.1 gram) were cut into small pieces (1 mm size) using a clean sharp blade, and transferred into 1.5 ml tubes. A 0.4 ml lysis buffer (20 mM Tris-HCl, pH 7.5, 150 mM NaCl, 1 mM Na₂EDTA, 1 mM EGTA, 1% Triton X-100, Protease inhibitor cocktail pill) was added into each sample tube. The tissues were homogenized using a Dounce homogenizer. After Homogenization, 50 μl of 10% SDS and 50 μl of 1M DTT were added into the mixture followed by incubation at 95° C. for 10 min. After incubation the extraction was sonicated to further breakdown DNA. Sonicated mixtures were centrifuged at 15,000×g for 10 minutes. Supernatants were collected and stored at −80° C. for further analysis. The protein concentration of the supernatants was determined by a BCA™ Reducing Reagent compatible assay kit (Pierce/Thermo Scientific). The expression levels of CAM1, CPA3, OLM4, LAD1, DPEP1, OGFR, EPHB3, PKP3, SERPINB5 and MUC13 proteins were determined by Mass Spectrometry. Or the amounts of CAM1, CPA3, OLM4, LAD1, DPEP1, OGFR, EPHB3, PKP3, SERPINB5 and MUC13 protein were used, the interaction between protein and its antibody was used as standard to determine the concentrations of biomarkers in the lysates from the normal tissues or colorectal tumors.

TABLE 5
Examples of the Expression of Biomarkers in normal
tissues and different stages of colorectal cancers

| | | Stage | Stage |
|---|---|---|---|
| Gene | Normal | TNM-I&II | TNM-III&IV |
| symbol | (ppm) | (ppm) | (ppm) |
| CEAM6 | 2.877 | <50 | >50 |
| EPHB3 | 1.916 | >15 | <15 |
| SERPINB5 | 9.854 | <150 | >150 |
| OLM4 | 74.397 | >300 | <300 |
| LAD1 | 1.762 | >50 | <50 |
| DPEP1 | 3.247 | >30 | <30 |

All of proteins in table 2 can be used as biomarkers for colorectal cancer. Any of the 740 proteins can be developed to a method useful or diagnostic kit for determining if a subject has an increased risk having a colorectal disease or disorder as disclosed in the present application. Applicant will claim the patent right for any patent resulting from the instant application, any continuations, divisions, re-issues, re-examinations and extensions thereof and corresponding patents and patent applications in other countries. Furthermore, all of biomarkers for colorectal cancer can be used for other tumors, such as bladder cancer, breast cancer, endometrial cancer, kidney cancer, colon cancer, leukemia, lung cancer, melanoma, non-Hodgkin lymphoma, pancreatic cancer, prostate cancer and thyroid cancer.

### REFERENCES

1. Network, C.G.A., Comprehensive molecular characterization of human colon and rectal cancer. Nature, 2012. 487(7407): p. 330-7.
2. Vogelstein, B., et al., Cancer genome landscapes. Science, 2013. 339(6127): p. 1546-58.
3. Schmitt, M. W., M. J. Prindle, and L. A. Loeb, Implications of genetic heterogeneity in cancer. Ann NY Acad Sci, 2012. 1267: p. 110-6.
4. Batlle, E., et al., Beta-catenin and TCF mediate cell positioning in the intestinal epithelium by controlling the expression of EphB/ephrinB. Cell, 2002. 111(2): p. 251-63.
5. Sancho, E., E. Batlle, and H. Clevers, Signaling pathways in intestinal development and cancer. Annu Rev Cell Dev Biol, 2004. 20: p. 695-723.
6. Zhang, B., et al., Proteogenomic characterization of human colon and rectal cancer. Nature, 2014. 513(7518): p. 382-7.
7. Besson, D., et al., A quantitative proteomic approach of the different stages of colorectal cancer establishes OLFM4 as a new nonmetastatic tumor marker. Mol Cell Proteomics, 2011. 10(12): p. M111.009712.
8. Han, C. L., et al., An informatics-assisted label-free approach for personalized tissue membrane proteomics: case study on colorectal cancer. Mol Cell Proteomics, 2011. 10(4): p. M110.003087.
9. Ballikaya, S., et al., De Novo Proteome Analysis of Genetically Modified Tumor Cells By a Metabolic Labeling/Azide-alkyne Cycloaddition Approach. Mol Cell Proteomics, 2014. 13(12): p. 3446-56.
10. Florens, L., et al., Analyzing chromatin remodeling complexes using shotgun proteomics and normalized spectral abundance factors. Methods, 2006. 40(4): p. 303-11.
11. Paoletti, A. C., et al., Quantitative proteomic analysis of distinct mammalian Mediator complexes using normalized spectral abundance factors. Proc Natl Acad Sci USA, 2006. 103(50): p. 18928-33.
12. Finnson, K. W., et al., Identification of CD109 as part of the TGF-beta receptor system in human keratinocytes. FASEB J, 2006. 20(9): p. 1525-7.
13. Botfield, H., et al., Decorin prevents the development of juvenile communicating hydrocephalus. Brain, 2013. 136(Pt 9): p. 2842-58.
14. Okamoto, O. and S. Fujiwara, Dermatopontin, a novel player in the biology of the extracellular matrix. Connect Tissue Res, 2006. 47(4): p. 177-89.
15. Wang, H., et al., Smad7 is inactivated through a direct physical interaction with the LIM protein Hic-5/ARA55. Oncogene, 2008. 27(54): p. 6791-805.
16. Moloney, D. J., et al., Fringe is a glycosyltransferase that modifies Notch. Nature, 2000. 406(6794): p. 369-75.
17. Frise, E., et al., The Drosophila Numb protein inhibits signaling of the Notch receptor during cell-cell interaction in sensory organ lineage. Proc Natl Acad Sci USA, 1996. 93(21): p. 11925-32.
18. Shimokawa, T., et al., Distinct roles of first exon variants of the tumor-suppressor Patched1 in Hedgehog signaling. Oncogene, 2007. 26(34): p. 4889-96.
19. Chi, S., et al., Rab23 negatively regulates Gli1 transcriptional factor in a Su(Fu)-dependent manner. Cell Signal, 2012. 24(6): p. 1222-8.
20. Kouzarides, T., Chromatin modifications and their function. Cell, 2007. 128(4): p. 693-705.
21. Lange, S. S., K. Takata, and R. D. Wood, DNA polymerases and cancer. Nat Rev Cancer, 2011. 11(2): p. 96-110.
22. Naba, A., et al., Extracellular matrix signatures of human primary metastatic colon cancers and their metastases to liver. BMC Cancer, 2014. 14: p. 518.
23. Bentires-Alj, M., et al., New methods in mammary gland development and cancer: proteomics, epigenetics, symmetric division and metastasis. Breast Cancer Res, 2012. 14(4): p. 314.
24. Kessenbrock, K., V. Plaks, and Z. Werb, Matrix metalloproteinases: regulators of the tumor microenvironment. Cell, 2010. 141(1): p. 52-67.
25. Murphy, D. A. and S. A. Courtneidge, The ‘ins’ and ‘outs’ of podosomes and invadopodia: characteristics, formation and function. Nat Rev Mol Cell Biol, 2011. 12(7): p. 413-26.
26. Krause, M. and A. Gautreau, Steering cell migration: lamellipodium dynamics and the regulation of directional persistence. Nat Rev Mol Cell Biol, 2014. 15(9): p. 577-90.
27. Sadanandam, A., et al., A colorectal cancer classification system that associates cellular phenotype and responses to therapy. Nat Med, 2013. 19(5): p. 619-25.
28. De Sousa E Melo, F., et al., Poor-prognosis colon cancer is defined by a molecularly distinct subtype and develops from serrated precursor lesions. Nat Med, 2013. 19(5): p. 614-8.
29. Langan, R. C., et al., Colorectal cancer biomarkers and the potential role of cancer stem cells. J Cancer, 2013. 4(3): p. 241-50.
30. Beck, M., et al., The quantitative proteome of a human cell line. Mol Syst Biol, 2011. 7: p. 549.

What is claimed is:

1. A method for determining if a subject has an increased risk having a colorectal disease or disorder comprising:
a) isolating a biological sample containing a test sample from a biopsy specimen from said subject,
b) isolating a biological sample containing normal colorectal cells or tissue from a biopsy specimen from said subject or a family member of said subject,
c) analyzing protein abundances of biomarkers for the samples from a) and b),
d) comparing the results from c) between the abnormal and normal colorectal cells or tissue.

2. The method of claim 1, wherein said colorectal disease or disorder is a colorectal cancer.

3. The method of claim 1, wherein said biomarkers is/are one or more proteins selected from the group consisting of CAM1, CPA3, OLM4, LAD1, DPEP1, OGFR, EPHB3, PKP3, CEAM6, SERPINB5 and MUC13 proteins.

4. The method of claim 3, wherein the expression of CAM1 and CPA3 proteins are lower in the abnormal than the normal colorectal cells or tissue, respectively.

5. The method of claim 3, wherein the expression of OLM4, LAD1, DPEP1, OGFR, EPHB3, PKP3, CEAM6, SERPINB5 and MUC13 proteins are higher in the abnormal than the normal colorectal cells or tissue, respectively.

6. The method of claim 1, wherein said subject is a human.

7. A kit for measuring the levels of biomarkers in a specimen, wherein the biomarkers are a panel of biomarkers and their measurable fragments.

8. The kit of claim 7, wherein said biomarkers is/are one or more proteins selected from the group consisting of CAM1, CPA3, OLM4, LAD1, DPEP1, OGFR, EPHB3, PKP3, CEAM6, SERPINB5 and MUC13 proteins.

9. The kit of claim 7, wherein the expression of CAM1 and CPA3 proteins are lower in the abnormal than the normal colorectal cells or tissue, respectively.

10. The kit of claim 7, wherein the expression of OLM4, LAD1, DPEP1, OGFR, EPHB3, PKP3, CEAM6, SERPINB5 and MUC13 proteins are higher in the abnormal than the normal colorectal cells or tissue, respectively.

11. The kit of claim 7, wherein the kit further includes standard proteins or peptides of the biomarkers or protein lysates from normal colorectal cells or/and colorectal cancer cells, antibodies against the biomarkers, processing reagents, support substances and detection reagents for the quantitation of the biomarkers.

12. The kit of claim 7, wherein the kit further includes normal colorectal tissues or/and colorectal cancer tissues, antibodies against the biomarkers, processing reagents, and detection reagents for the quantitation of the biomarkers.
